SEQUENCE LISTING (3 sequences)

SEQ ID NO: 1 - RNA, 21 nt, Synthetic construct
  source 1..21: mol_type = other RNA
tgagctctgtgttgaccttga

SEQ ID NO: 2 - RNA, 21 nt, Synthetic construct
  source 1..21: mol_type = other RNA
tccctggcaagtggcattcat

SEQ ID NO: 3 - RNA, 21 nt, Synthetic construct
  source 1..21: mol_type = other RNA
cctggactatgtggccagcaa

(19) United States
(12) Patent Application Publication
(10) Pub. No.: US 2026/0201390 A1
(43) Pub. Date: Jul. 16, 2026

(54) GENE THERAPY FOR LEVODOPA-INDUCED DYSKINESIA IN PARKINSONS DISEASE

(71) Applicant: Northwestern University, Evanston, IL (US)

(72) Inventors: Dalton James SURMEIER, JR., Evanston, IL (US); Tatiana TKATCH, Evanston, IL (US); Shenyu ZHAI, Evanston, IL (US)

(21) Appl. No.: 19/451,120

(22) Filed: Jan. 16, 2026

Related U.S. Application Data
(60) Provisional application No. 63/746,116, filed on Jan. 16, 2025.
(60) Provisional application No. 63/748,822, filed on Jan. 23, 2025.

(51) Int. Cl. C12N 15/113; A61K 9/00; A61K 31/197; A61P 25/16; C12N 15/86

(52) U.S. Cl. CPC C12N 15/1138; A61K 9/0019; A61K 31/197; A61P 25/16; C12N 15/86; C12N 2310/14; C12N 2310/531; C12N 2750/14143

(57) ABSTRACT

The present disclosure provides compositions and methods for the treatment of levodopa-induced dyskinesia (LID) and Parkinson's disease (PD) therewith. In particular, the present disclosure provides methods for treating LID in PD by inhibiting (e.g., expression of) Muscarinic M1 acetylcholine receptor (M1Rs), for example, in indirect pathway spiny projection neurons (iSPNs) to attenuate LID without compromising of the treatment with levodopa (e.g., boosting the efficacy of levodopa treatment).

### CROSS-REFERENCE

This application claims the benefit of U.S. Provisional Patent Application No. 63/746,116, filed Jan. 16, 2025, and U.S. Provisional Patent Application No. 63/748,822, filed Jan. 23, 2025, both of which are incorporated by reference herein in its entirety.

### STATEMENT REGARDING FEDERALLY SPONSORED RESEARCH OR DEVELOPMENT

This invention was made with government support under grant number NS034696 awarded by the National Institutes of Health. The government has certain rights in the invention.

### SEQUENCE LISTING

The text of the computer readable sequence listing filed herewith, titled “NWEST-44244_203_SequenceListing.xml”, created Jan. 16, 2026, having a file size of 3,712 bytes, is hereby incorporated by reference in its entirety.

### FIELD

The present disclosure provides compositions and methods for the treatment of levodopa-induced dyskinesia (LID) and Parkinson's disease (PD) therewith. In particular, the present disclosure provides methods for treating LID and PD by inhibiting (e.g., expression of) Muscarinic M1 acetylcholine receptor (M1Rs), for example, in indirect pathway spiny projection neurons (iSPNs) to attenuate LID without compromising the treatment with levodopa (e.g., boosting the efficacy of levodopa treatment).

### BACKGROUND

Parkinson's disease (PD) is the second most common neurodegenerative disorder. In the United States alone, more than one million Americans are living with PD and this number is projected to increase to 1.2 million by 2030 due to the aging population. PD is characterized as a progressive neurological disorder that primarily affects movement, causing tremors, stiffness, and difficulty with balance and coordination.

One prominent aspect of PD is the dual motor and non-motor symptomology that arises from the loss of dopamine-producing neurons in the substantia nigra region of the brain. As a result, non-motor symptoms include cognitive changes such as memory difficulties, slowed thinking, and executive dysfunction, with some patients developing PD dementia. In terms of motor symptoms, movement is significantly affected in PD due to the degeneration of dopamine-producing neurons which are critical for motor control. This is because dopamine is the key neurotransmitter in the facilitation and communication between the substantia nigra and the striatum, which are essential regions within the basal ganglia for coordinating smooth and controlled muscle movements. Therefore, the progressive loss of these dopamine-producing neurons leads to a deficiency of dopamine, disrupting the normal signaling pathways within the basal ganglia. This disruption results in the hallmark motor symptoms of PD, including tremors, bradykinesia (slowness of movement), rigidity (muscle stiffness), and postural instability (balance problems). Without adequate dopamine, the basal ganglia cannot properly regulate motor functions, leading to the characteristic difficulties in initiating and controlling movements.

In the early stages of PD, when a substantial population of dopaminergic neurons remain, boosting their release of dopamine (DA) by administration of its blood-brain barrier penetrant precursor, levodopa (L-3,4-dihydroxyphenylalanine), effectively alleviates motor symptoms. However, as the disease progresses, higher oral doses of levodopa are required to achieve symptomatic benefit and as a result, brain concentrations of DA become dysregulated—rising to abnormally high levels for hours and then falling back to very low levels as systemic levodopa wains. As the disease progresses and dopaminergic neurons continue to decline, patients often experience fluctuations in their response to levodopa, leading to motor complications. One such complication is levodopa-induced dyskinesia (LID), which are involuntary, erratic, and often writhing movements that can occur as a complication of long-term levodopa use. Dyskinesias typically emerge during the later stages of the disease, reflecting the cumulative impact of prolonged dopaminergic therapy and disease progression. Management of LID may involve adjusting the dosage and timing of levodopa, adding other medications to smooth out the response, or considering advanced therapies such as deep brain stimulation (DBS) which involves invasive surgery. Nevertheless, adjusting levodopa or introducing new medications to a PD patient can cause a host of issues such as, fluctuations and worsening of motor control, increased side effects, and the potential for new complications. Likewise, the effectiveness of DBS can vary significantly among patients, and this variability makes it difficult to predict outcomes for individual patients. In some instances, DBS may even exacerbate these non-motor issues, and the surgical risks include potential complications such as infection, bleeding, and stroke, which, although rare, can be serious. In the long term, DBS does not stop the progression of Parkinson's disease, and patients may develop new symptoms that are unresponsive to the treatment.

Therefore, there is a critical need for treatments that effectively address LID in Parkinson's disease without introducing additional deficits or interfering with the efficacy of levodopa. Such treatments would enhance motor control and quality of life for patients by managing dyskinesias while preserving the benefits of existing dopaminergic therapies. This would ensure that patients maintain optimal symptom control with minimal side effects, facilitating better long-term management of the disease.

### SUMMARY

Provided herein are compositions and methods for the treatment of levodopa-induced dyskinesia (LID) and Parkinson's disease (PD) therewith. In particular, the present disclosure provides methods for treating LID in PD by inhibiting (e.g., expression of) Muscarinic M1 acetylcholine receptor (M1Rs), for example, in indirect pathway spiny projection neurons (iSPNs) to attenuate LID without compromising the treatment with levodopa (e.g., boosting the efficacy of levodopa treatment).

Embodiments of the present disclosure include a method for treating Levodopa-induced dyskinesia (LID), in a subject in need thereof, the method comprising: administering a M1 muscarinic acetylcholine receptor (M1R) antagonist to the subject.

In some embodiments, the M1R antagonist is administered into an indirect pathway spiny projection neuron (iSPN).

In some embodiments, the M1R antagonist selectively disrupts M1 muscarinic acetylcholine receptor signaling.

In some embodiments, the M1R antagonist is a nucleic acid inhibitor of M1 muscarinic acetylcholine expression.

In some embodiments, the nucleic acid inhibitor of M1 muscarinic acetylcholine expression knocks down the expression of iSPN M1Rs to suppress cholinergic signaling to attenuate oscillations in dendritic excitability and synaptic strength.

In some embodiments, the nucleic acid inhibitor of M1 muscarinic acetylcholine expression is selected from a microRNA (miRNA), a small interfering (siRNA), a short hairpin RNA (shRNA), an anti-sense RNA (asRNA), a competing endogenous RNA (ceRNA), a long non-coding RNA (lncRNA) and a ribozyme. In some embodiments, the nucleic acid inhibitor of M1 muscarinic acetylcholine expression is an shRNA having at least 70% (e.g., 70%, 75%, 80%, 85%, 90%, 95%, 100%) sequence identity to SEQ ID NO: 3.

In some embodiments, the nucleic acid inhibitor of M1 muscarinic acetylcholine expression is delivered with a vector.

In some embodiments, the vector is a viral vector.

In some embodiments, the viral vector is an adeno-associated viral vector (AAV), a adenoviral vector, a lentiviral vector, a non-viral vector, a human-compatible vector, a herpes simplex virus vector and a retroviral vector.

In some embodiments, the subject is afflicted with the neurodegenerative disease associated with intrinsic and synaptic changes in striatal spiny projection neurons (SPNs) triggered by the sustained elevation of dopamine (DA).

In some embodiments, the neurodegenerative disease is Parkinson's disease.

In some embodiments, the subject is a human.

In some embodiments, the subject was administered or is currently being administered a therapeutic agent.

In some embodiments, the therapeutic agent administered is levodopa (L-3,4-dihydroxyphenylalanine).

In some embodiments, wherein administering levodopa to the subject induces fluctuations of DA levels in a subject; wherein large and uncontrolled fluctuations in DA levels results in LID.

In some embodiments, wherein administering levodopa to the subject increases acetylcholine release in the subject's off-state.

In some embodiments, wherein the increased acetylcholine release in the off-state results in the upregulation of oscillations in dendritic excitability and synaptic strength.

In some embodiments, wherein the M1R antagonist reduces the function of iSPN M1Rs to diminish the induction and expression of LID.

In some embodiments, wherein the M1R antagonist is administered by an Intraperitoneal (IP) injection.

Embodiments of the present disclosure include a method for treating PD comprising administering an antagonist of M1R.

Embodiments of the present disclosure include a method for treating PD comprising administering levodopa and an antagonist of M1R.

In some embodiments, wherein said M1R antagonist is administered repeatedly to the subject.

In some embodiments, wherein said M1R antagonist is administered once to the subject.

In some embodiments, wherein said M1R antagonist is administered during the low levels of DA to the subject after levodopa is administered.

Embodiments of the present disclosure include a M1R antagonist for use in the manufacture of a medicament for the treatment of a neurodegenerative disease in a subject in need thereof.

Embodiments of the present disclosure include a M1R antagonist for use in the treatment of a neurodegenerative disease in a subject in need thereof.

Embodiments of the present disclosure include a M1R antagonist for use in the manufacture of a medicament for the treatment of LID in a subject in need thereof.

Embodiments of the present disclosure include a M1R antagonist for use in the treatment of LID in a subject in need thereof.

### BRIEF DESCRIPTION OF THE DRAWINGS

FIGS. 1A-I. dSPN somatic and dendritic excitability changed between off- and on-states in LID mice. (A) Experimental timeline. LID mice were sacrificed 24-48 hours or 30 min after the last levodopa injection for ex vivo assessment of the off-state and on-state, respectively. (B) Confocal image of a coronal brain slice from a unilaterally 6-OHDA lesioned, LID on-state mouse immunostained with anti-phospho-ERK (pERK, red) and anti-tyrosine hydroxylase (TH, green) antibodies. Scale bar, 0.5 mm. (C) Schematic illustrating the somatic excitability assay in dSPNs. (D) Sample voltage recordings from dSPNs in the off-state and on-state of LID in response to a 200-pA current injection. Scale bars, 10 mV and 200 ms. (E) Current-response curves showing that somatic excitability of dSPNs was state-dependent (control, n=10 cells from 6 mice; 6-OHDA, n=12 cells from 5 mice; off-state, n=15 cells from 8 mice; on-state, n=15 cells from 10 mice; on-state+SCH, n=10 cells from 5 mice). (F) Box plot summary of rheobase in dSPNs. ****p<0.0001, Mann-Whitney test. (G) 2PLSM image of a patched dSPN with imaging sites on proximal and distal dendrites indicated. Scale bar, 20 μm. (H) Current injections of 2 nA (top), are aligned with evoked somatic spikes (middle) and fluorescence transients recorded from proximal and distal dendrites (bottom). Scale bars, 25 mV, 0.1 ΔG/R₀ and 0.5 s. (I) Box plot summary of dendritic excitability index (the distal to proximal ratio of area under the curve (AUC) of ΔG/R₀) of dSPNs (control, n=11 cells from 6 mice; 6-OHDA, n=9 cells from 5 mice; off-state, n=10 cells from 7 mice; on-state, n=10 cells from 5 mice; on-state+SCH, n=9 cells from 5 mice). *p<0.05, Mann-Whitney test.

FIGS. 2A-I. dSPN spine morphology and unitary synaptic strength changed between off- and on-states in LID mice. (A) Left, a low-magnification 2PLSM image of a patched dSPN with its dendritic tree visualized by Alexa dye and its proximal and distal locations used for spine density measurements delineated. Scale bar is 20 μm. Right, sample 2PLSM images of dendritic segments of dSPNs. Scale bar represents 5 μm. (B-C) Box plot summaries of density of all dendritic spines (B) and density of mushroom-type spines (C) in proximal and distal dendrites of dSPNs (control, n=10 cells from 6 mice; 6-OHDA, n=11 cells from 5 mice; off-state, n=13 cells from 8 mice; on-state, n=10-11 cells from 7-8 mice). *p<0.05, **p<0.01, ***p<0.0001, Mann-Whitney test. (D) Confocal image of ChR2-YFP expression in the motor cortex in a coronal section. Scale bar is 1 mm. (E) Schematic illustrating the experimental setup for Sr²⁺-oEPSC measurement. Whole-cell patch clamp recordings were made from dSPNs in acute brain slices of mice and oEPSCs were evoked by brief blue LED pulses. (F) Sample traces of Sr²⁺-oEPSC evoked by optogenetic stimulation of cortical afferents (indicated by blue vertical lines) in the presence of 3 mM Sr²⁺ and nominally 0 Ca²⁺ from dSPNs in LID off- and on-states. Scale bars denote 20 pA and 100 ms. (G) Cumulative probability plot of Sr²⁺-oEPSC amplitudes in dSPNs from off-state and on-state mice. (H-I) Box plots showing an increase in Sr²⁺-oEPSC amplitudes (H) in on-state dSPNs, but not in Sr²⁺-oEPSC frequency (I) (off-state, n=10 cells from 4 mice; on-state, n=8 cells from 4 mice). ***p<0.001, n.s. not statistically significant, Mann-Whitney test.

FIGS. 3A-F. Somatic and dendritic excitability of iSPNs changed between off- and on-states in LID mice. (A) Schematic illustrating the somatic excitability assay in iSPNs. (B) Sample voltage recordings from off-state and on-state iSPNs in response to a 200-pA current injection (500 ms duration). Scale bars are 10 mV and 200 ms. (C) Current-response curves showing the decrease in iSPN somatic excitability in the on-state compared to off-state (control, n=21 cells from 10 mice; 6-OHDA, n=11 cells from 6 mice; off-state, n=12 cells from 5 mice; on-state, n=11 cells from 8 mice; on-state+D2R antagonist, n=7 cells from 4 mice). (D) Box plot summary of rheobase in iSPNs. ***p<0.001, **p<0.01, Mann-Whitney test. (E) Left, schematic illustrating the dendritic excitability assay in iSPNs. Right, Fluo-4 fluorescence transients (bottom) recorded from iSPN proximal and distal dendrites are aligned with current injections of 2 nA (top) and corresponding somatic spikes (middle). Scale bars denote 25 mV, 0.1 ΔG/R₀ and 0.5 s. (F) Box plot summary of dendritic excitability index (the distal to proximal ratio of AUC of ΔG/R₀) in iSPNs from control, 6-OHDA lesioned, off-state, on-state or on-state mice with bath application of sulpiride (control, n=14 cells with 8 mice; 6-OHDA, n=9 cells from 6 mice; off-state, n=10 cells from 5 mice; on-state, n=10 cells from 5 mice; on-state+sul, n=8 cells from 6 mice). **p<0.01, *p<0.05, Mann-Whitney test.

FIGS. 4A-K. Spine morphology and unitary synaptic strength of iSPNs changed between off- and on-states in LID mice. (A) Left, 2PLSM image of a patched iSPN with its proximal and distal dendritic segments delineated. Scale bar, 20 μm. Right, 2PLSM images of iSPN dendrites. Scale bar, 5 μm. (B-C) Box plot summaries of total (B) and mushroom (C) spine densities in proximal and distal dendrites of iSPNs (control, n=15 cells from 9 mice; 6-OHDA, n=11 cells from 6 mice; off-state, n=9 cells from 5 mice; on-state, n=9 cells from 5 mice). (D) Schematic illustrating the experimental setup for Sr²⁺-oEPSC measurement. (E) Representative recordings of Sr²⁺-oEPSC evoked by optogenetic stimulation of cortical afferents (indicated by blue vertical lines) from iSPNs of off-state and on-state mice. Scale bars, 20 pA and 100 ms. (F) Cumulative probability plot of Sr²⁺-oEPSC amplitudes. (G-H) Box plot summaries of Sr²⁺-oEPSC amplitudes (G) and frequencies (H) in iSPNs (off-state, n=7 cells from 4 mice; on-state, n=8 cells from 4 mice). (I) Confocal image of sparsely labeled iSPNs in a control mouse. Scale bar, 100 μm. (J) Representative high-resolution confocal images showing proximal dendrites of sparsely labeled iSPNs. Scale bar, 3 μm. (K) Box plot summary of spine density in proximal dendrites of iSPNs imaged by high-resolution confocal microscopy (control, n=18 dendrites from 4 mice; 6-OHDA, n=22 dendrites from 3 mice; off-state, n=25 dendrites from 4 mice; on-state, n=22 dendrites from 4 mice). (L) Schematic showing different distributions of iSPN spine size in control, 6-OHDA lesioned, off-state and on-state mice, as well as the detection thresholds of 2PLSM and confocal microscopy. ****p<0.0001, ***p<0.001, *p<0.05, n.s. not significant, Mann-Whitney test.

FIGS. 5A-F. ACh release was increased in the striata of PD and LID mice but strongly suppressed by DA. (A) Diagram showing injection of GRABACh3.0-expressing AAV into the DLS of mice. (B) Pseudocolor images of GRABACh3.0 signal (ΔF/F₀) in resting state (‘rest’) and immediately following stimulation (‘stim’) in the DLS of unlesioned (‘control’) or 6-OHDA lesioned (‘6-OHDA’) mice. Scale bar is 20 μm. (C-E) Representative traces of the fluorescence response of GRABACh3.0 signals in the DLS of control (C), 6-OHDA lesioned (D), and LID off-state mice (E) evoked by an intrastriatal electrical stimulation before and after bath application of D2R agonist(s) (quinpirole or DA) or D2R antagonist sulpiride. Scale bars are 1 s and 0.2 ΔF/F₀. (F) Box plot summary of GRABACh3.0 signal (AUC of ΔF/(Fₘₐₓ−Fₘᵢₙ)) in unlesioned control, 6-OHDA lesioned, and LID off-state mice. In 6-OHDA lesioned and off-state mice, ACh release was significantly elevated. Bath application of DA (50 nM), mimicking the high DA condition in LID on-state, strongly suppressed ACh release (unlesioned control, n=13 regions of interest (ROIs) from 3 mice; 6-OHDA, n=10 ROIs from 3 mice; off-state, n=11 ROIs from 5 mice). *p<0.05, **p<0.01, ***p<0.001, ****p<0.0001, Mann-Whitney (unpaired) and Wilcoxon (paired) tests.

FIGS. 6A-F. M1R mediated the dendritic, but not the somatic, alterations in iSPNs in LID-off state. (A) Schematic illustrating the somatic excitability assay in iSPNs from LID off-state mice in which M1R signaling was inhibited throughout the off-state. (B) Sample voltage changes evoked by 200-pA current injections (500-ms duration) in iSPNs from off-state mice that received M1R antagonist (“+M1R ant.”) or vehicle control throughout the off-state. Scale bars are 10 mV and 200 ms. (C) Current-response curves showing that iSPN somatic excitability in off-state was resistant to M1R antagonists (off-state control, n=8 cells from 4 mice; off-state with M1R antagonists, n=10 cells from 5 mice). Historical off-state data from FIG. 3C was overlaid as a dashed line. (D) Left, schematic illustrating the dendritic excitability assay in iSPNs. Right, summary of dendritic excitability index in iSPNs from off-state mice treated with M1R antagonist or vehicle control (off-state control, n=9 cells from 5 mice; off-state+M1R antagonists, n=7 cells from 4 mice). *p<0.05, Mann-Whitney test. (E) Left, low-magnification image showing an iSPN from an off-state mouse treated with M1R antagonists, patched and filled with Alexa dye. Scale bar is 20 μm. Right, sample 2PLSM images of dendritic segments of iSPNs from off-state mice treated with vehicle (‘control’) or M1R antagonists (‘+M1R antagonist’). Scale bar is 5 μm. (F) Box plot summary of dendritic spine density in off-state iSPNs treated with vehicle or M1R antagonists (control, n=8 cells from 4 mice; +M1R antagonist, n=9 cells from 5 mice). ***p<0.001, Mann Whitney test. Historical on-state data from FIG. 4B was shown by a dashed line.

FIGS. 7A-H. CDGI signaling pathway mediated the dendritic changes in off-state iSPNs. (A) Schematic of somatic excitability assay. (B) Sample voltage recordings from on-state and off-state iSPNs from CDGI KO mice. Scale bars, 10 mV and 200 ms. (C) Current-response curves of on- and off-state iSPNs from CDGI KO (on-state, n=8 cells from 4 mice; off-state, n=13 cells from 5 mice). (D) Left, schematic of dendritic excitability assay. Right, Fluo-4 signals from iSPN proximal and distal dendrites before and after oxo-M application in slices from wildtype mice. Current injections (2 nA) and somatic spikes were shown on top. Scale bars, 25 mV, 50% ΔF/F₀ and 0.5 s. (E) Box plot summary showing the effect of oxo-M on iSPN dendritic excitability in wildtype and CDGI KO mice (n=10-12 dendrites from 3 mice per genotype). **p<0.01, n.s., no significance. Wilcoxon test. (F) Box plot summary of dendritic excitability index of iSPNs from wildtype or CDGI KO mice in off- or on-state (wildtype data were from FIG. 3F; KO off-state, n=11 cells from 5 mice; KO on-state, n=8 cells from 4 mice). **p<0.01, n.s., no significance, Mann-Whitney test. (G) Left, image of a patched iSPN from an off-state CDGI KO mouse. Scale bar, 20 μm. Right, images of iSPN dendrites from CDGI KO mice. Scale bar, 5 μm. (H) Box plot summary of iSPN spine density in on- and off-state CDGI KO mice (on-state, n=8 cells from 4 mice; off-state, n=11 cells from 6 mice). n.s. not statistically significant, Mann-Whitney test.

FIG. 8A-C. Genetic deletion of CDGI enhanced the motoric effect of levodopa and attenuated dyskinetic behaviors. (A) Box plot summary of the number of contralateral rotations (in 30 seconds) recorded 40 min after the fifth levodopa administration in wildtype or CDGI KO mice (wildtype n=12 animals; CDGI KO, n=10 animals). *p<0.05, Mann-Whitney test. (B-C) Plots of total (B), axial, limb and orolingual (C) AIM scores as a function of sessions in wildtype and CDGI KO mice (wildtype n=12 mice; CDGI KO, n=11 mice; data are mean±SEM). Total AIM score: time p<0.001, F(4, 84)=17.19; group p<0.001, F(1, 21)=41.87. Axial score: time p<0.001, F(2.429, 51)=11.32; group p=0.002, F(1, 21)=19.91. Limb score: time p<0.001, F(3.019, 63.40)=13.00; group p<0.001, F(1, 21)=25.65. Orolingual score: time p<0.001, F(3.192, 67.04)=13.84; group p<0.001, F(1, 21)=56.29. Repeated measure two-way ANOVA and post-hoc Bonferroni test was used.

FIGS. 9A-F. FIG. 9. Deletion of M1R from iSPNs prevented dendritic changes in the off-state. (A) Left, experimental timeline for 6-OHDA lesioning, M1R CRISPR expression, AIM testing and ex vivo recordings. Right, schematic illustrating injection of M1R CRISPR into the DLS of Adora2-Cre mice. (B) Confocal image showing expression of gRNA-FusionRed in the DLS of a coronal section. Scale bar is 1 mm. (C) Sample somatic voltage changes in response to 200-pA current injections in iSPNs from off-state mice without or with M1R deletion from iSPNs. Scale bars are 10 mV and 200 ms. (D) Current-response curves showing that the increase in iSPN somatic excitability in the off-state was not prevented by M1R deletion (control: n=6 cells from 3 mice; M1R deletion: n=8 cells from 4 mice). Historical off-state data from FIG. 3C was shown by a dashed line. (E) Left, low-magnification image showing a patched iSPN visualized by Alexa dye and expression of gRNA-FusionRed in nearby cells in a non-specific manner. Cell type-specific deletion was achieved by Cre-dependent expression of saCas9 in Adora2-Cre mice. Scale bar is 20 μm. Right, 2PLSM images of dendritic segments of iSPNs from off-state mice, without or with M1R deletion. Scale bar indicates 5 μm. (F) Box plot summary of dendritic spine density of iSPNs without or with M1R deletion from off-state mice (control: n=6 cells from 3 mice; M1R deletion: n=10 cells from 4 mice). ***p<0.001, Mann-Whitney test. Historical on-state data from FIG. 4B was indicated by a dashed line.

FIGS. 10A-F. Deletion of M1R from iSPNs attenuated dyskinetic behaviors and enhanced the motoric effect of levodopa. (A) Box plot summary of the number of contralateral rotations (in 30 seconds) recorded 40 min after the fifth levodopa administration (control n=11 animals; M1R deletion, n=10 animals). (B) Plots of total, axial, limb and orolingual AIM scores as a function of sessions in mice without or with iSPN-specific deletion of M1R. Genetic deletion of M1R in iSPNs produced an overall reduction in AIM scores (control, n=11 animals; M1R CRISPR, n=10 animals; data are mean±SEM). Total AIM score: time p<0.001, F(3.313, 62.95)=22.18; group p<0.001, F(1, 19)=29.30. Axial score: time p<0.001, F(2.562, 48.67)=21.80; group p=0.0015, F(1, 19)=13.80. Limb score: time p<0.001, F(3.144, 59.74)=17.01; group p<0.001, F(1, 19)=41.43. Orolingual score: time p<0.001, F(3.595, 68.31)=12.42; group p<0.001, F(1, 19)=44.58. Repeated measure two-way ANOVA followed by post-hoc Bonferroni test. (C) Top, the experimental timeline for M1R CRISPR expression and open field experiments with MCI-Park mice. Bottom, schematic illustrating bilateral injection of M1R CRISPR into the DLS of MCI-Park mice. (D) Representative traces of locomotor activity in the open field test. (E-F) Box plots of distance traveled (E) and time mobile (F) in 5 min (control, n=12 mice; MCI-Park, n=17 mice; MCI-Park with M1R deletion, n=9 mice; MCI-Park with levodopa, n=12 mice; MCI-Park with both M1R deletion and levodopa, n=9 mice). ****, p<0.0001, ***, p<0.001, *p<0.05, Mann-Whitney test.

FIGS. 11A-B. The frequency of Sr²⁺-oEPSC was unaltered between off- and on-states in both dSPNs and iSPNs. (A) A representative box plot summary of Sr²⁺-oEPSC frequency in off- and on-state dSPNs (off-state, n=10 cells from 4 mice; on-state, n=8 cells from 4 mice). n.s., not statistically significant, Mann-Whitney test. (B) A representative box plot summary of Sr²⁺-oEPSC frequency in off-state and on-state iSPNs (off-state, n=7 cells from 4 mice; on-state, n=8 cells from 4 mice). n.s., not statistically significant, Mann-Whitney test.

FIG. 12. ACh release evoked by burst stimulation was elevated in 6-OHDA lesioned and LID off-state mice and suppressed by D2R agonists. Box plot summary of GRABACh3.0 signals evoked by burst stimulation in unlesioned, 6-OHDA lesioned and LID off-state mice. Same as with single stimulation, ACh release evoked by burst stimulation (20 pulses at 20 Hz) was significantly elevated in 6-OHDA lesioned and LID off-state mice. Bath application of DA (50 nM) or quinpirole (10 mM) strongly suppressed ACh release (unlesioned control, n=13 ROIs from 3 mice; 6-OHDA, n=10 ROIs from 3 mice; off-state, n=11 ROIs from 5 mice). ****p<0.0001, ***p<0.001, **p<0.01, n.s., not statistically significant, Mann-Whitney test for unpaired data and Wilcoxon for paired data.

FIGS. 13A-B. Genetic perturbation of M1R-CDGI signaling in iSPNs increases the therapeutic effect of levodopa in parkinsonian mice. (A) Box plot summary of the number of contralateral rotations (in 30 s) recorded after the first and fifth levodopa administration in wildtype or CDGI KO mice (wildtype n=11-12 animals; CDGI KO, n=9-10 animals). The session 5 data were the same as in FIG. 8A. *p<0.05, n.s. not statistically significant, Mann-Whitney test. (B) Box plot summary of the number of contralateral rotations (in 30 s) recorded after the first or fifth levodopa administration in control or M1R CRISPR mice (control n=11 animals; M1R CRISPR, n=10 animals). The session 5 data were the same as in FIG. 10A. **p<0.01, n.s. not statistically significant, Mann-Whitney test.

FIGS. 14A-C. Functional validation of M1R CRISPR. (A) Example traces of somatic voltage recordings in response to a 180-pA current injection from iSPNs expressing M1R CRISPR or gRNA alone (control) before and after application of oxo-M. Scale bars are 40 mV and 100 ms. (B) Box plot summary of the effect of oxo-M on the number of action potentials (APs) evoked (control, n=7 cells from 3 mice; M1R CRISPR, n=6 cells from 3 mice). The increase in somatic excitability by oxo-M was prevented by M1R CRISPR. *p<0.05, Mann-Whitney test. (C) Box plot summary of the effect of oxo-M on rheobase. *p<0.05, Mann-Whitney test.

FIG. 15. Signaling model of cell type-specific changes in SPNs in the on- and off-states of levodopa-induced dyskinesia (LID). Insets, illustration of signaling pathways underlying state-dependent synaptic plasticity. Black arrowheads indicate positive regulation and black circles indicate negative regulation. AC5, adenylyl cyclase type 5; AMPAR, a-amino-3-hydroxyl-5-methyl-4-isoxazole-propionic acid receptor; Kv1, Kv1 voltage-dependent potassium channel); Kv4, Kv4 voltage-dependent potassium channel); Kv7, Kv7 voltage-dependent potassium channels); NMDAR, N-methyl-D-aspartate receptor PKA, protein kinase A; SK, small-conductance Ca²⁺-activated potassium channel.

FIG. 16. Density of spines with larger head diameters detected by high-resolution confocal microscopy. Box plot summary of the density of spines with >0.4 mm head diameter in proximal dendrites of sparsely labeled iSPNs imaged by high-resolution confocal microscopy (control: n=18 dendrites from 4 mice; 6-OHDA: n=22 dendrites from 3 mice; off-state: n=25 dendrites from 4 mice;

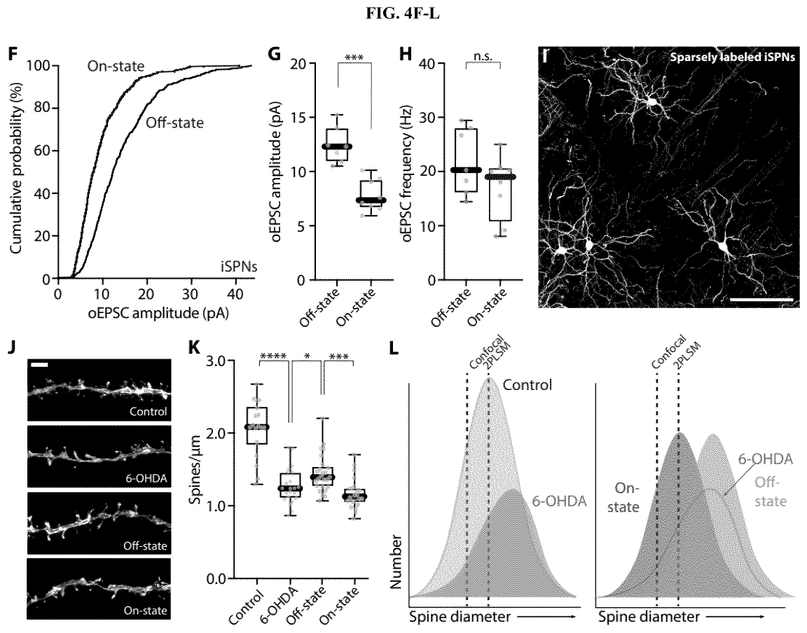

on-state: n=22 dendrites from 4 mice). This result was similar to the total spine density detected by 2PLSM (FIG. 4B). ***p<0.001, **p<0.01, *p<0.05, Mann-Whitney test.

### Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise.

The present disclosure also contemplates other embodiments “comprising,” “consisting of” and “consisting essentially of,” the embodiments or elements presented herein, whether explicitly set forth or not.

As used herein, the term “effective amount” refers to the amount of a composition (e.g., pharmaceutical composition) sufficient to effect beneficial or desired results. An effective amount can be administered in one or more administrations, applications or dosages and is not intended to be limited to a particular formulation or administration route.

As used herein and in the appended claims, the singular forms “a”, “an” and “the” include plural reference unless the context clearly dictates otherwise. Thus, for example, reference to “a M1R” is a reference to one or more a M1R, unless the context clearly dictates otherwise.

As used herein, the term “administering” refers to the act of giving a drug, prodrug, or other agent, or therapeutic treatment (e.g., pharmaceutical compositions of the present invention) to a subject or in vivo, in vitro, or ex vivo cells, tissues, and organs. Exemplary routes of administration to the human body can be through the eyes (e.g., intraocularly, intravitreally, periocularly, ophthalmic, etc.), mouth (oral), skin (transdermal), nose (nasal), lungs (inhalant), oral mucosa (buccal), ear, rectal, by injection (e.g., intravenously, subcutaneously, intratumorally, intraperitoneally, etc.) and the like.

As used herein, the terms “antagonist” and “inhibitor” are used interchangeably and refer to any agent that interferes with or diminishes the normal expression or activity of a receptor, enzyme, or other molecular target. This antagonism/inhibition can result in the partial reduction or complete prevention of the expression and/or activity of the target. The terms encompass a wide range of molecular entities, including but not limited to, small molecules, peptides, antibodies, aptamers, and nucleic acids (e.g., shRNA), all of which can disrupt the normal expression and/or function of the target and alter cellular or organismal responses.

As used herein, the term “comprise”, and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for 30 ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

As used herein, the term “shRNA” (small hairpin RNA) refers to an RNA duplex wherein a portion of the RNA is part of a hairpin structure (shRNA). In addition to the duplex portion, the hairpin structure may contain a loop portion positioned between the two sequences that form the duplex. The loop can vary in length. In some embodiments, the loop is 5, 6, 7, 8, 9, 10, 11, 12 or 13 nucleotides in length. The hairpin structure can also contain 3′ or 5′ overhang portions. In some aspects, the overhang is a 3′ or a 5′ overhang 0, 1, 2, 3, 4 or 5 nucleotides in length. In one aspect of this invention, a nucleotide sequence in the vector serves as a template for the expression of a small hairpin RNA, comprising a sense region, a loop region and an antisense region. Following expression, the sense and antisense regions form a duplex. It is this duplex, forming the shRNA, which hybridizes to a target mRNA and reduces expression thereof.

As used herein, the term “knock-down” or “knock-down technology” refers to a technique of gene silencing in which the expression of a target gene or gene of interest is reduced as compared to the gene expression prior to the introduction of the siRNA, which can lead to the inhibition of production of the target gene product.

As used herein, the terms “treatment,” “treating,” and the like refer to obtaining a desired pharmacologic and/or physiologic effect against a particular disease, disorder, or condition. Preferably, the effect is therapeutic, i.e., the effect partially or completely cures the disease and/or adverse symptom attributable to the disease. The terms “treatment” and “treating” refers to reversing, alleviating, slowing down, inhibiting the progression of, preventing, or reducing the likelihood of the disease, disorder, or condition to which such term applies, or one or more symptoms or manifestations of such disease, disorder, or condition. In some embodiments, for example, the terms “treatment” and “treating” of a disease also includes providing relief from the symptoms or side-effects of the disease (including palliative treatment).

As used herein, the term “pharmaceutical composition” refers to the combination of an active agent with a carrier, inert or active, making the composition especially suitable for diagnostic or therapeutic use in vitro, in vivo or ex vivo.

As sed herein, the terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, 10 water, emulsions (e.g., such as an oil/water or water/oil emulsions), and various types of wetting agents, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers, and adjuvants, see, e.g., Martin, Remington's Pharmaceutical Sciences, 15th Ed., 15 Mack Publ. Co., Easton, Pa. (1975), incorporated herein by reference in its entirety.

### DETAILED DESCRIPTION

The present disclosure provides compositions and methods for the treatment of levodopa-induced dyskinesia (LID) and Parkinson's disease (PD) therewith. In particular, the present disclosure provides methods for treating LID in PD by inhibiting (e.g., expression of) Muscarinic M1 acetylcholine receptor (M1Rs), for example, in indirect pathway spiny projection neurons (iSPNs) to attenuate LID without compromising of the treatment with levodopa (e.g., boosting the efficacy of levodopa treatment).

In Parkinson's disease (PD), the loss of the dopaminergic neurons in the substantia nigra pars compacta (SNc) disrupts basal ganglia circuitry, leading to bradykinesia, rigidity and tremor. In the early stages of PD, systemic levodopa administration boosts the production and release of dopamine (DA), effectively ameliorating motor symptoms. However, as the disease progresses, the levodopa dose needed to achieve symptomatic benefits rises and the machinery regulating extracellular DA wanes in efficacy. As a result, DA signaling is dysregulated, rising dramatically for hours after levodopa treatment and then falling to very low levels until the next dose is taken. This abnormal, slow oscillation in brain DA concentration triggers alterations in basal ganglia circuits that result in uncontrolled movements (i.e., dyskinesia) shortly after taking levodopa. Although several parts of the basal ganglia have been implicated in the emergence of LID, there is a consensus that the striatum is a critical site of pathophysiology.

The principal neurons of the striatum are GABAergic spiny projection neurons (SPNs), which constitute ~90% of all striatal neurons. About half of SPNs—direct pathway SPNs (dSPNs)—project directly to the output nuclei of the basal ganglia, promoting action selection. The other half—the iSPNs—project to the external segment of the globus pallidus and thus are indirectly connected to the output nuclei; activity in iSPNs suppress contextually inappropriate actions. Due to their differential expression of DA receptors, iSPNs and dSPNs are modulated by DA in opposite ways. In dSPNs, Gs/olf-coupled D1 DA receptors (DIRs) stimulate adenylyl cyclase (AC) and protein kinase A (PKA), increasing intrinsic excitability, enhancing glutamatergic synaptic transmission, and facilitating long-term synaptic potentiation (LTP). In contrast, in iSPNs, Gi-coupled D2 DA receptors (D2Rs) inhibit AC and stimulate phospholipase C, decreasing intrinsic excitability, attenuating glutamatergic transmission, and promoting long-term synaptic depression (LTD). Importantly, in the healthy striatum, dopaminergic signaling is episodic, being linked to the initiation of actions and to the outcomes of actions. These transient signaling events are thought to be critical to connecting actions and their outcomes to modifications in axospinous synaptic strength that underlie the acquisition of habits and contextually appropriate goal-directed actions.

The impact of dopaminergic signaling on SPNs is normally modulated by a dynamic interaction with autonomously active, giant, cholinergic interneurons (ChIs). Acting through D2Rs, striatal DA release inhibits the autonomous spiking of ChIs and their release of acetylcholine (ACh). The ACh released by ChIs acts on iSPNs and dSPNs in ways that complement those of DA. In dSPNs, which primarily express Gi-coupled M4 muscarinic receptors (M4Rs), ACh signaling blunts the effects of DIR activation and promotes LTD induction, whereas, in iSPNs, which only express M1 muscarinic receptors (M1Rs), ACh signaling enhances somatic excitability, dendritic integration and LTP induction. The M1R-mediated modulation of iSPN dendrites is dependent upon CalDAG-GEFI (CDGI), a striatum-enriched, Ca²⁺ activated guanine nucleotide exchange factor. Thus, the interaction between dopaminergic and cholinergic signaling not only modulates the moment-to-moment excitability of striatal ensembles coordinating purposeful movement, but also long-term changes in synaptic strength that guide future behavior.

In models of late-stage PD, where the vast majority of dopaminergic neurons innervating the striatum have been lost, there appears to be a sustained enhancement of ACh release by ChIs. This shift is attributable to an elevation in ChI intrinsic excitability, a strengthening of their excitatory glutamatergic input from the parafascicular nucleus, and a dis-inhibition of ACh release from ChI terminals. In animal models of PD, optogenetic or chemogenetic inhibition of ChIs alleviates motor deficits. Furthermore, optogenetic activation of ChIs in healthy mice induces a parkinsonian-like state. The hypothesis that elevated ACh release contributes to the hypokinetic features of PD is also supported by the clinical observation that muscarinic receptor antagonists are of symptomatic benefit. But, the involvement of ChIs in the dyskinesia induced by levodopa treatment is controversial. On one hand, boosting cholinergic signaling appears to attenuate LID. For example, enhancing M4R signaling in dSPNs attenuates LID severity. On the other hand, several studies suggest that ChI ablation or disruption of cholinergic signaling attenuates LID severity. One critical gap in these studies is an assessment of how cholinergic signaling and SPNs are changing between the period when mice are dyskinetic and striatal DA is high (on-state) and when mice are hypokinetic and striatal DA is low (off-state). It is highly likely that the activity of ChIs and cholinergic signaling in these two states are very different. Moreover, as DA and ACh normally work together to control long-term changes in the functional connectomes of SPN underlying learning, it could be that the aberrant interaction between these neuromodulators induced by high doses of levodopa lead to pathological ‘learning’ and alterations in circuitry that carry over from one state to the next.

To help fill this fundamental gap in understanding, a combination of electrophysiological, imaging, genetic, pharmacological and behavioral approaches were employed in a mouse model of LID. These studies revealed that LID on- and off-states were associated with bidirectional, cell-type specific changes in intrinsic excitability and synaptic connectivity. In addition, these studies demonstrated that ACh release by ChIs was elevated in the parkinsonian and LID off-states. Moreover, the release of ACh by ChIs continued to be negatively modulated by D2Rs in tissue from dyskinetic mice, arguing that striatal cholinergic signaling was inhibited in the on-state. The dysregulation of cholinergic signaling was critical to the state-dependent alterations in synaptic strength and dendritic spine architecture in iSPNs. Blunting the impact of ChIs on iSPNs not only attenuated dyskinetic behaviors, it enhanced the beneficial effects of levodopa treatment. Taken together, systemic administration of an M1R antagonist blunted the induction of LID and boosted levodopa's therapeutic benefits. More specifically, cell-type specific knockdown of M1Rs using viral delivery of an shRNA diminished LID and boosted the efficacy of levodopa treatment.

Embodiments of the present disclosure include a method for treating Levodopa-induced dyskinesia (LID), in a subject in need thereof, the method comprising: administering a M1 muscarinic acetylcholine receptor (M1R) antagonist to the subject.

In some embodiments, the M1R antagonist is administered into an indirect pathway spiny projection neuron (iSPN).

In some embodiments, the M1R antagonist selectively disrupts M1 muscarinic acetylcholine receptor signaling.

In some embodiments, M1R antagonist is a nucleic acid inhibitor of M1 muscarinic acetylcholine expression.

In some embodiments, the nucleic acid inhibitor of M1 muscarinic acetylcholine expression knocks down the expression of iSPN M1Rs to suppress cholinergic signaling to attenuate oscillations in dendritic excitability and synaptic strength.

In some embodiments, the nucleic acid inhibitor of M1 muscarinic acetylcholine expression is selected from a microRNA (miRNA), a small interfering (siRNA), a short hairpin RNA (shRNA), an anti-sense RNA (asRNA), a competing endogenous RNA (ceRNA), a long non-coding RNA (lncRNA) and a ribozyme. In some embodiments, the nucleic acid inhibitor of M1 muscarinic acetylcholine expression is delivered with a vector. In some embodiments, the vector is a viral vector. In some embodiments, the viral vector is an adeno-associated viral vector (AAV), a adenoviral vector, a lentiviral vector, a non-viral vector, a human-compatible vector, a herpes simplex virus vector and a retroviral vector.

In some embodiments, the nucleic acid inhibitor of M1 muscarinic acetylcholine expression (i.e., M1R antagonist) is an siRNA. In some embodiments, a siRNA is an 18 to 30 nucleotide, preferably 19 to 25 nucleotide, most preferred 21 to 23 nucleotide or even more preferably 21 nucleotide-long double-stranded RNA molecule. siRNAs naturally found in nature have a well-defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest. Essentially any gene for which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, in some embodiments at least one RNA strand has a 5′- and/or 3′-overhang. Preferably, one end of the double-strand has a 3′-overhang from 1 to 5 nucleotides, more preferably from 1 to 3 nucleotides and most preferably 2 nucleotides. The other end may be blunt-ended or has up to 6 nucleotides 3′-overhang. In some embodiments, siRNA duplexes are provided composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair. 2′-deoxynucleotides in the 3′ overhangs are as efficient as ribonucleotides but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as 5% glucose) or cationic lipids and polymers can be used for siRNA delivery in vivo through systemic routes either intravenously (IV) or intraperitoneally (IP).

In some embodiments, the nucleic acid inhibitor of M1 muscarinic acetylcholine expression (i.e., M1R antagonist) is a short hairpin RNA (shRNA). In some embodiments, the si/shRNAs to be used in the present invention are chemically synthesized using appropriately protected ribonucleoside phosphoramidites and a conventional DNA/RNA synthesizer. In some embodiments, provided herein are shRNA molecules that target and inhibit the expression (e.g., knockdown) of the M1R.

Further molecules effecting RNAi (and useful herein for the inhibition of expression of the M1R) include, for example, microRNAs (miRNA). Said RNA species are single-stranded RNA molecules. Endogenously present miRNA molecules regulate gene expression by binding to a complementary mRNA transcript and triggering the degradation of said mRNA transcript through a process similar to RNA interference. Accordingly, exogenous miRNA may be employed as an antagonist of M1R after introduction into target cells. In some embodiments, provided herein are miRNA molecules that target and inhibit the expression (e.g., knock down) of the M1R.

Morpholinos (or morpholino oligonucleotides) are synthetic nucleic acid molecules having a length of about 20 to 30 nucleotides and, typically about 25 nucleotides. Morpholinos bind to complementary sequences of target transcripts by standard nucleic acid base-pairing. They have standard nucleic acid bases which are bound to morpholine rings instead of deoxyribose rings and linked through phosphorodiamidate groups instead of phosphates. Due to replacement of anionic phosphates into the uncharged phosphorodiamidate groups, ionization in the usual physiological pH range is prevented, so that morpholinos in organisms or cells are uncharged molecules. The entire backbone of a morpholino is made from these modified subunits. Unlike inhibitory small RNA molecules, morpholinos do not degrade their target RNA molecules. Rather, they sterically block binding to a target sequence within a RNA and prevent access by molecules that might otherwise interact with the RNA. In some embodiments, provided herein are morpholino oligonucleotides that target and inhibit the expression (e.g., knockdown) of M1R.

A ribozyme (ribonucleic acid enzyme, also called RNA enzyme or catalytic RNA) is an RNA molecule that catalyzes a chemical reaction. Many natural ribozymes catalyze either their own cleavage or the cleavage of other RNAs, but they have also been found to catalyze the aminotransferase activity of the ribosome. Non-limiting examples of well-characterized small self-cleaving RNAs are the hammerhead, hairpin, hepatitis delta virus, and in vitro-selected lead-dependent ribozymes, whereas the group I intron is an example for larger ribozymes. The principle of catalytic self-cleavage is well established. Since it was shown that hammerhead structures can be integrated into heterologous RNA sequences and that ribozyme activity can thereby be transferred to these molecules, catalytic antisense sequences can be engineered for almost any target sequence that can be created, provided the target sequence contains a potential matching cleavage site. The basic principle of constructing hammerhead ribozymes is as follows: A region of interest of the RNA, which contains the GUC (or CUC) triplet, is selected. Two oligonucleotide strands, each usually with 6 to 8 nucleotides, are taken and the catalytic hammerhead sequence is inserted between them. In some embodiments, provided herein are ribozyme inhibitors or oligonucleotides of M1R.

In some embodiments, M1R is inhibited (and/or M1R activity is inhibited) by modifying the M1R sequence in target cells. In some embodiments, the alteration of M1R is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). Exemplary guide RNAs that find use in embodiments herein for disrupting M1R expression are SEQ ID NOS: 1 and 2. Alternative guide RNA sequences are within the scope herein. One or more elements of a CRISPR system can derive from a type I, type II, or type III CRISPR system, e.g., derived from a particular organism comprising an endogenous CRISPR system, such as Streptococcus pyogenes. In some aspects, a Cas nuclease and gRNA (including a fusion of crRNA specific for the target sequence (e.g., a sequence within M1R) and fixed tracrRNA) are introduced into the cell. In general, target sites at the 5′ end of the gRNA target the Cas nuclease to the target site, e.g., M1R, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence (e.g., sequence within M1R). In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex. The CRISPR system can induce double stranded breaks (DSBs) at the SRC-3 target site, followed by disruptions or alterations as discussed herein. In other embodiments, Cas9 variants, deemed “nickases,” are used to nick a single strand at the target site (e.g., within M1R). Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression (e.g., to inhibit expression of M1R). In some embodiments, the CRISPR system is used to alter M1R and/or inhibit expression of M1R.

In some embodiments, the subject is afflicted with the neurodegenerative disease associated with intrinsic and synaptic changes in striatal spiny projection neurons (SPNs) triggered by the sustained depletion of dopamine (DA).

In some embodiments, the neurodegenerative disease is Parkinson's disease.

In some embodiments, the subject is a human.

In some embodiments, the subject was administered or is currently being administered a therapeutic agent.

In some embodiments, the therapeutic agent administered is levodopa (L-3,4-dihydroxyphenylalanine).

Levodopa (L-DOPA) or L-3,4-dihydroxyphenylalanine: is a chemical that is made and used as part of the normal biology of humans, some animals, and plants. Levodopa is the precursor to the neurotransmitters dopamine, norepinephrine (noradrenaline), and epinephrine (adrenaline) collectively known as catecholamines. Levodopa mediates neurotrophic factor release by the brain and CNS. Levodopa is manufactured and sold as a psychoactive drug under trade names including SINEMET, PHARMACOPA, ATAMET, STALEVO, MADOPAR, and PROLOPA. Levodopa is prescribed and administered for the treatment of neurodegenerative disorders, and in particular in the clinical treatment of Parkinson's disease.

In other embodiments, administering levodopa to the subject induces fluctuations of DA levels in a subject; wherein large and uncontrolled fluctuations in DA levels results in LID.

In some embodiments, levodopa is initially administered to a subject at a dose of 200-600 mg. In some embodiments, levodopa is initially administered to a subject at a dose of 200 mg. In some embodiments, levodopa is initially administered to a subject at a dose of 250 mg. In some embodiments, levodopa is initially administered to a subject at a dose of 300 mg. In some embodiments, levodopa is initially administered to a subject at a dose of 350 mg. In some embodiments, levodopa is initially administered to a subject at a dose of 400 mg. In some embodiments, levodopa is initially administered to a subject at a dose of 450 mg. In some embodiments, levodopa is initially administered to a subject at a dose of 500 mg. In some embodiments, levodopa is initially administered to a subject at a dose of 550 mg. In some embodiments, levodopa is initially administered to a subject at a dose of 600 mg.

In some embodiments, levodopa is administered to a to a subject once a day. In some embodiments, levodopa is administered to a to a subject twice a day. In some embodiments, levodopa is administration is increased over time.

In some embodiments, levodopa is administered to a subject at a maintained dose of 2000 to 7000 mg/day. In some embodiments, levodopa is administered to a subject at a maintained dose of 2000 mg/day. In some embodiments, levodopa is administered to a subject at a maintained dose of 2500 mg/day. In some embodiments, levodopa is administered to a subject at a maintained dose of 3000 mg/day. In some embodiments, levodopa is administered to a subject at a maintained dose of 3500 mg/day. In some embodiments, levodopa is administered to a subject at a maintained dose of 4000 mg/day. In some embodiments, levodopa is administered to a subject at a maintained dose of 4500 mg/day. In some embodiments, levodopa is administered to a subject at a maintained dose of 5000 mg/day. In some embodiments, levodopa is administered to a subject at a maintained dose of 5500 mg/day. In some embodiments, levodopa is administered to a subject at a maintained dose of 6000 mg/day. In some embodiments, levodopa is administered to a subject at a maintained dose of 6500 mg/day. In some embodiments, levodopa is administered to a subject at a maintained dose of 7000 mg/day.

In some embodiments, levodopa is administered to a subject at a maintained dose into three separate dosages. In some embodiments, levodopa administration does not exceed a dose of 8000 mg/day.

In some embodiments, administering levodopa to the subject increases acetylcholine release in the subject's off-state.

In some embodiments, the increased acetylcholine release in the off-state results in the upregulation of oscillations in dendritic excitability and synaptic strength.

In some embodiments, the M1R antagonist reduces the function of iSPN M1Rs to diminish the induction and expression of LID.

In some embodiments, the M1R antagonist is administered by an Intraperitoneal (IP) injection.

Embodiments of the present disclosure include a method for treating PD comprising administering an antagonist of M1R.

Embodiments of the present disclosure include a method for treating PD comprising administering levodopa and an antagonist of M1R.

In some embodiments, said M1R antagonist is administered repeatedly to the subject.

In some embodiments, said M1R antagonist is administered once to the subject.

In some embodiments, said M1R antagonist is administered during the low levels of DA to the subject after levodopa is administered.

Embodiments of the present disclosure include a M1R antagonist for use in the manufacture of a medicament for the treatment of a neurodegenerative disease in a subject in need thereof.

Embodiments of the present disclosure include a M1R antagonist for use in the treatment of a neurodegenerative disease in a subject in need thereof.

Embodiments of the present disclosure include a M1R antagonist for use in the manufacture of a medicament for the treatment of LID in a subject in need thereof.

Embodiments of the present disclosure include a M1R antagonist for use in the treatment of LID in a subject in need thereof.

As is well known in the medical arts, dosages (e.g., levodopa dosages, etc.) for any one patient depends upon many factors, including the patient's size, body surface area, age, the particular compound to be administered, sex, time and route of administration, general health, and interaction with other drugs being concurrently administered.

Pharmaceutical compositions herein may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration.

For injection, pharmaceutical compositions may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art.

In other embodiments, the pharmaceutical compositions are formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions, and the like, for oral or nasal ingestion by a patient to be treated.

Pharmaceutical compositions include compositions wherein the active ingredients (e.g., levodopa, etc.) are contained in an effective amount to achieve the intended purpose. For example, an effective amount of therapeutic may be an amount that prevents LID and/or treats or reduces symptoms associated with PD. Determination of effective amounts is well within the capability of those skilled in the art, especially in light of the disclosure provided herein.

In addition to the active therapeutic ingredients, pharmaceutical compositions may contain suitable pharmaceutically acceptable carriers comprising excipients and auxiliaries that facilitate processing of the active compounds into preparations that can be used pharmaceutically. The preparations formulated for oral administration may be in the form of tablets, dragees, capsules, or solutions. The pharmaceutical compositions of the present invention may be manufactured in a manner that is itself known (e.g., by means of conventional mixing, dissolving, granulating, dragee-making, levigating, emulsifying, encapsulating, entrapping, or lyophilizing processes).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc; cellulose such as methyl cellulose, hydroxypropyl methyl-cellulose, or sodium carboxymethylcellulose; and gums including arabic and tragacanth; and proteins such as gelatin and collagen. If desired, disintegrating, or solubilizing agents may be added, such as the cross-linked polyvinyl pyrrolidone, agar, alginic acid or a salt thereof such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, (e.g., dosage).

Pharmaceutical preparations for oral administration include push-fit capsules made of gelatin, as well as soft, sealed capsules made of gelatin and a coating such as glycerol or sorbitol. The push-fit capsules can contain the active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Therapeutic compositions formulated in a pharmaceutical acceptable carrier may be prepared, placed in an appropriate container, and labeled for treatment of the indicated condition (e.g., levodopa-induced dyskinesias, PD, etc.).

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

In some embodiments, a therapeutically effective dose may be estimated initially from cell culture assays and/or animal models (particularly murine models). A therapeutically effective dose refers to that amount that effectively addresses and underlying cause and/or ameliorates symptoms of the disease state or unwanted condition (e.g., levodopa-induced dyskinesias, PD, etc.). Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. Data obtained from these cell culture assays, and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration. The exact dosage is chosen by the individual clinician in view of the patient to be treated. Dosage and administration are adjusted to provide sufficient levels of the active moiety or to maintain the desired effect. Additional factors which may be taken into account include the severity of the disease state; age, weight, and gender of the patient; diet, time and frequency of administration, drug combination(s), reaction sensitivities, and tolerance/response to therapy. Long-acting pharmaceutical compositions might be administered every 3 to 4 days, every week, or once every two weeks depending on half-life and clearance rate of the particular formulation.

Typical dosage amounts may vary from 0.1 to 100,000 micrograms, up to a total dose of about 1 g, depending upon the route of administration. Guidance as to particular dosages and methods of delivery is provided in the literature (See, U.S. Pat. Nos. 4,657,760; 5,206,344; 5,225,212; WO2004/097009, or WO2005/075465, each of which are herein incorporated by reference).

In some embodiments, the therapies disclosed herein are combined or used in combination with other agents useful in the treatment of psychomotor diseases (e.g., PD). Or, by way of example only, the therapeutic effectiveness of one of the therapies described herein may be enhanced by administration of an adjuvant (e.g., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously, or sequentially with a compound as disclosed herein. When a compound as disclosed herein is used contemporaneously with one or more other drugs, a pharmaceutical composition containing such other drugs in addition to the compound disclosed herein may be utilized but is not required.

In some embodiments, one or more of the therapies provided herein (e.g., levodopa, etc.) are combined with each other, and/or with one or more treatments for a psychomotor disease (e.g., PD). Suitable treatments for psychomotor disease (e.g., PD) for co-administration (e.g., with levodopa, etc.) include dopamine agonists (e.g., bromocriptine, pergolide, pramipexole, ropinirole, piribedil, cabergoline, apomorphine, lisuride, etc.), MAO-B inhibitors (e.g., selegiline, rasagiline, etc.), and other therapeutics, such as amantadine, anticholinergics, quetiapine, cholinesterase inhibitors, modafinil, non-steroidal anti-inflammatory drugs, etc.

In some embodiments, deep brain stimulation is also utilized for the treatment of PD, in addition to the other embodiments described herein.

In some embodiments, one or more therapeutic approaches described herein co-administered to a subject. In some embodiments, co-administration involves co-formulation of two or more agents together into the same medicament. In other embodiments, the agents are in separate formulations but are administered together, either simultaneously or in sequence (e.g., separated by one or more minutes, hours, days, etc.). In some embodiments, where a synergistic or additive benefit is achieved, the co-administered agent may be provided at a lower dose than would normally be administered if that agent were being used in isolation to treat the disease or condition.

### EXPERIMENTAL

The following Materials/Methods and Examples are offered as illustrative as a partial scope and particular embodiments of the disclosure and are not meant to be limiting of the scope of the disclosure.

Animals. Animal use procedures were approved by the Northwestern Institutional Animal Care and Use committee. Male C57Bl/6 hemizygous mice (7-12 weeks of age) expressing tdTomato or eGFP under control of Drd1a or Drd2 receptor regulator elements (RRID: MMRRC_030512-UNC and RRID: MMRRC_000230-UNC, backcrossed to C57BL/6 background) were used. In some experiments, these mice were crossed with CDGI KO mice in a C57BL/6 J background (Crittenden et al. 2021; Crittenden et al. 2009). BAC transgenic mice expressing Cre recombinase under control of the A2aR regulatory elements (RRID: MMRRC 031168-UCD) were used for high-resolution confocal microscopy and M1R CRISPR experiments.

Unilateral 6-OHDA model of PD and LID. Mice were anesthetized using an isoflurane precision vaporizer (at 5% isofluorane during induction and 2% isofluorane during maintenance phase) and positioned in a stereotaxic frame (David Kopf Instruments, model 940). Mice were administered with analgesics meloxicam (METACAM®, 0.1 mg/kg, s.c., Covetrus) before surgery. After the skin and fascia were retracted to reveal the skull, a small hole was drilled over the MFB. 3.5 mg ml-1free base 6-OHDA hydrochloride (Sigma Aldrich, Cat. No. H4381) freshly dissolved in saline with 0.02% L-ascorbic acid (Sigma Aldrich, Cat. No. A92902) was injected using a glass calibrated micropipette (Drummond Scientific Company, Broomall, PA, 200005) at the following coordinates: AP (mm relative to Bregma): −0.7; ML: −1.2; DV: −4.75 (from dura). Mice were monitored daily post-op and supplemented with saline injections and high fat/high sucrose food as needed (Shen et al. 2024). Three to four weeks after surgery, the degree of lesioning of nigrostriatal DA neurons was assessed with a drug-free cylinder test (Fieblinger et al. 2014; Shen et al. 2024). Within 1-7 days of the cylinder test, mice underwent behavioral testing for abnormal involuntary movements (AIMs) as previously described (Cenci et al. 2007; Fieblinger et al. 2014; Shen et al. 2024). In brief, mice were transferred to a behavioral testing room, placed in clean cage bottoms without bedding, and administered intraperitoneal injections of L-DOPA at 6 mg/kg for the first two sessions and 12 mg/kg for the later sessions. Benserazide was co-administered at 12 mg/kg to inhibit peripheral conversion of levodopa to DA. Behavioral testing was performed every other day for a total of five test sessions. AIMs (axial, limb, and orolingual movements) were scored as previously described (Cenci et al. 2007; Fieblinger et al. 2014; Shen et al. 2024): abnormal axial, limb, and orolingual behaviors were observed for one minute every 20 min and rated on a scale from 0-4 for each parameter on the basis of duration and continuity. Mice were sacrificed for ex vivo experiments 24-48 hours (off-state) or 0.5 hour (on-state) after the last levodopa administration. Striatal and nigral sections from a subset of mice were stained with tyrosine hydroxylase to verify successful lesion.

Viral injections. To study synaptic responses at corticostriatal synapses, 0.15 μl AAV5-hSyn-hChR2(H134R)-eYFP (Addgene #26973) was injected into the M1 motor cortex ipsilateral to the lesion site at the following coordinate (mm relative to Bregma): ML: −1.60, AP: 1.15, DV: 1.55. To sparsely label iSPNs for high-resolution confocal imaging of dendritic spines, Adora2-Cre mice (~2 months old) were injected with AAV9-pCAG-flex-eGFP-WPRE (Addgene #51502) (0.450 μl, titer of 5×10¹⁰ viral genome/ml) into the left DLS at (mm relative to Bregma): AP: 0.7, ML: −2.35, DV: ~3.35. To express the ACh sensor GRABACh3.0 in the striatum, 0.6 μl of AAV5-hSyn-ACh3.0 (ACh4.3) (WZ Biosciences, YL001003-AV5) was injected into the DLS ipsilateral to the 6-OHDA lesion at the following coordinate (mm relative to Bregma): ML: −2.2, AP: 0.8, DV: 3.3. For M1R gene deletion using CRISPR-Cas9, well-lesioned animals were randomly assigned to receive viral injection of either a mixture of two viruses (at 1:1 ratio): AAV9-hSyn-DIO-saCAS-minWPRE and AAV9-U6-gRNA (M1R)-U6-gRNA2-hSyn-FusionRed (M1R CRISPR, custom made by Virotek), or a mixture of saline and the gRNA virus (control). The viral injection (0.5 μl) was in the DLS (ipsilateral to the 6-OHDA lesion) at a total of 6 sites at the following coordinates (mm relative to Bregma): AP: 0.9, ML: −2.3, DV: −3.4 and −2.8; AP: 0.6, ML: −1.5, DV: −3.4 and −2.8; AP: 0.24, ML: −1.9, DV: −3.3 and −2.7. For ex vivo experiments that required identification of CRISPR-expressing iSPNs, a third virus: AAV9-EF1a-DIO-eYFP (Addgene #27056) was mixed with M1R CRSIPR or control (at 1:1:1 ratio) and injected. All experiments were performed about 3 weeks (for Cre-independent expression) or 4-5 weeks (for Cre-dependent expression) after viral injections.

Slice electrophysiology. Mice were deeply anesthetized with a mixture of ketamine (100 mg/kg) and xylazine (7 mg/kg) and perfused transcardially with ice-cold sucrose-based cutting solution containing (in mM): 181 sucrose, 25 NaHCO₃, 1.25 NaH₂PO₄, 2.5 KCl, 0.5 CaCl₂), 7 MgCl₂, 11.6 sodium ascorbate, 3.1 sodium pyruvate and 5 glucose (305 mOsm/1). Sagittal slices (280-μm thick) were sectioned using a vibratome (Leica VT1200). After cutting, slices were incubated at 34° C. for 30 minutes in ACSF containing (in mM): 124 NaCl, 3 KCl, 1 NaH₂PO₄, 2.0 CaCl₂, 1.0 MgCl₂, 26 NaHCO₃ and 13.89 glucose, after which they were stored at room temperature until recording. External solutions were oxygenated with carbogen (95% CO2/5% O2) at all time.

Individual slices were transferred to a recording chamber and continuously superfused with ACSF (2-3 ml/min, 31-32° C.). D1-Tdtomato- or D2-eGFP-expressing SPNs in the striatum were first identified with an Olympus BX-51-based two-photon laser scanning microscope (Ultima, Bruker). Whole-cell patch clamp was then performed in identified SPNs, aided by visualization with a 60×/0.9NA water-dipping objective lens and a ½″ CCD video camera (Hitachi) imaged through a Dodt contrast tube and a 2× magnification changer (Bruker). For somatic, dendritic, and morphological experiments, patch pipettes (3-4 MΩ resistance) were loaded with internal solution containing (mM): 115 K-gluconate, 20 KCl, 1.5 MgCl₂, 5 HEPES, 0.2 EGTA, 2 Mg-ATP, 0.5 Na-GTP, 10 Na-phosphocreatine (pH 7.25, osmolarity 280-290 mOsm/L). Cells were recorded in the current-clamp configuration. For Sr²⁺-oEPSC experiments, patch pipettes (3-4 M (2 resistance) were loaded with 120 CsMeSO₃, 5 NaCl, 0.25 EGTA, 10 HEPES, 4 Mg-ATP, 0.3 Na-GTP, 10 TEA, 5 QX-314 (pH 7.25, osmolarity 280-290 mOsm/L). SPNs were held at −70 mV in the voltage-clamp configuration. After patching, recording solution was changed to Ca²⁺-free ACSF containing 3 mM SrCl₂ and 10 μM gabazine (10 μM, to suppress GABAA-mediated currents). Slices were incubated with this Ca²⁺-free solution for 25 min before recording. EPSCs were evoked every 30 s by whole-field LED illumination (single 0.3-ms pulses). All the electrophysiological recordings were made using a MultiClamp 700B amplifier (Axon Instrument, USA), and signals were filtered at 2 kHz and digitized at 10 kHz. Voltage protocols and data acquisition were performed by Praire View 5.3 (Bruker). The amplifier command voltage and all light source shutter and modulator signals were sent via the PCI-NI6713 analog-to-digital converter card (National Instruments, Austin, TX).

Imaging of spines and dendritic Ca²⁺ transients with two-photon laser scanning microscopy (2PLSM). The pipette solution (EGTA omitted) was supplemented with 100 μM Fluo-4 (Thermo Fisher Scientific, F14200) and 50 μM Alexa Fluor 568 hydrazide (Thermo Fisher Scientific, A10437). After whole-cell recording configuration was established, cells were allowed to equilibrate with dyes for at least 15 min before imaging. The recorded SPN was visualized using 810 nm excitation laser (Chameleon Ultra II, Coherent, Santa Clara, USA). Dendritic structure was visualized by the red signal of Alexa Fluor 568 detected by a Hamamatsu R3982 side-on photomultiplier tube (PMT, 580-620 nm). Calcium transients, as signals in the green channel, were detected by a Hamamatsu H7422P-40 GaAsP PMT (490-560 nm, Hamamatsu Photonics, Japan). Signals from both channels were background subtracted before analysis. Line scan signals were acquired with 128 pixels per line resolution and 10 μs/pixel dwell time along a dendritic segment. Ca²⁺ signals were quantified as the area of increase in green fluorescence from baseline normalized by the average red fluorescence (ΔG/R) or the average baseline green fluorescence (ΔF/F₀). Dendritic excitability was measured as the ratio of the Ca²⁺ signal from a distal location to the Ca²⁺ signal from a proximal location on the same dendrite. Only data with similar baseline levels (Go/R₀) for proximal and distal locations were included.

For assessment of dendritic spine density, images of dendritic segments (proximal: ~40 μm from soma; distal: >80 μm from soma) were acquired with 0.15 μm pixels with 0.3 μm z-steps. Images were deconvolved in AutoQuant X3.0.4 (MediaCybernetics, Rockville, MD) and semi-automated spine counting was performed using 3D reconstructions in NeuronStudio (CNIC, Mount Sinai School of Medicine, New York). On average, two proximal and two distal dendrites were imaged and analyzed per neuron.

Two-photon imaging of ACh sensor. ACh release was assessed by imaging GRABACh3.0, a genetically encoded fluorescent sensor of Ach (Jing et al. 2020), using 2PLSM. Acute slices with striatal expression of GRABACh3.0 were prepared as described above, transferred to a recording chamber, and continuously perfused with normal ACSF at 32-34° C. A two-photon laser (Chameleon Ultra II, Coherent, Santa Clara, CA) tuned to 920 nm was used to excite GRABACh3.0. Fluorescence was imaged using an Ultima In Vitro Multiphoton Microscope system (Bruker, Billerica, MA) with an Olympus 60×/0.9 NA water-immersion objective lens and a Hamamatsu H7422P-40 GaAsP PMT (490 nm to 560 nm, Hamamatsu Photonics, Hamamatsu, Japan). Time series images of the GRABACh3.0 were acquired with 0.388 μm×0.388 μm pixels, 8-μs dwell time and a frame rate of 21.26 fps. After 3-seconds baseline acquisition, synchronous ACh release was evoked by delivering a single (1 ms×0.3 mA) or a train of 20 electrical stimuli (20 Hz) by a concentric bipolar electrode (CBAPD75, FHC) placed at 200 μm ventral to the region of interest. Imaging was continued for at least another 5 seconds. Two trials were performed for each stimulation protocol and data averaged. The minimal (Fₘᵢₙ) and maximal fluorescence intensity (Fₘₐₓ) were determined by applying 10 μM TTX (to block any basal transmission) and 100 μM acetylcholine chloride (to saturate ACh3.0 signal), respectively. The whole image was the region of interest (ROI) used for analysis. Fluorescent intensity data were analyzed by custom Python code (accessible upon request). Briefly, the fluorescence intensity values were first background-subtracted (the background resulted from PMT was measured by imaging with same PMT voltage but zero laser power). Baseline fluorescence F₀ was the average fluorescence over the 1 seconds period right before stimulation. ΔF=F−F₀ was normalized by (Fₘₐₓ−Fₘᵢₙ) and then analyzed.

High-resolution confocal microscopy of sparsely labeled neurons. Adora2-Cre mice injected with Cre-dependent eGFP virus and treated with 4 different conditions (control, 6-OHDA, LID off-state, LID on-state) were anesthetized with isoflurane followed by ketamine/xylazine mixture. LID on-state mice received a sixth dose of levodopa (12 mg/kg, supplemented with 12 mg/kg benserazide) one hour before anesthesia. The mice were transcardially perfused with 1× phosphate buffered saline (PBS, ~20 ml) followed by 4% paraformaldehyde (PFA, ~30 ml). Brains were then dissected out, postfixed in 4% PFA for 1.5-2 hrs, transferred to 1×PBS with 0.1% sodium azide and stored at 4° C. until sectioning. 60-μm sagittal sections containing the DLS were cut using a Leica VT1200S vibratome. Sections were mounted onto glass slides with No. 1.5 cover glasses using ProLong™ Diamond Antifade Mountant (Thermo Fisher Scientific, Cat. No. P36961).

Data acquisition, analysis and statistics. Electrophysiology and imaging data were acquired using PCI-NI6052E analog-to-digital converter card (National Instruments) and Praire View 5.3 (Bruker). Off-line analyses of electrophysiology data (except Sr2-oEPSCs), calcium imaging data and time-series imaging data were performed using custom-written Python scripts (available upon request). Amplitudes of Sr2-0EPSCs were analyzed automatically using TaroTools Event Detection in Igor Pro 8 (WaveMetrics, Portland, Oregon) followed by visual verification. Events were measured between 40 ms to 400 ms after photo stimulation. The threshold for detection of an event was greater than 5 SD above the noise. All summary data were presented as non-parametric box-whisker plots and statistical analyses performed by Prism 6 (GraphPad). The stated n indicates the number of cells (in electrophysiology experiments) or the number of mice (in AIMs testing). Comparisons were made using Mann-Whitney test (unpaired) or Wilcoxon test (paired) and differences with p<0.05 are considered statistically significant.

### Example 1

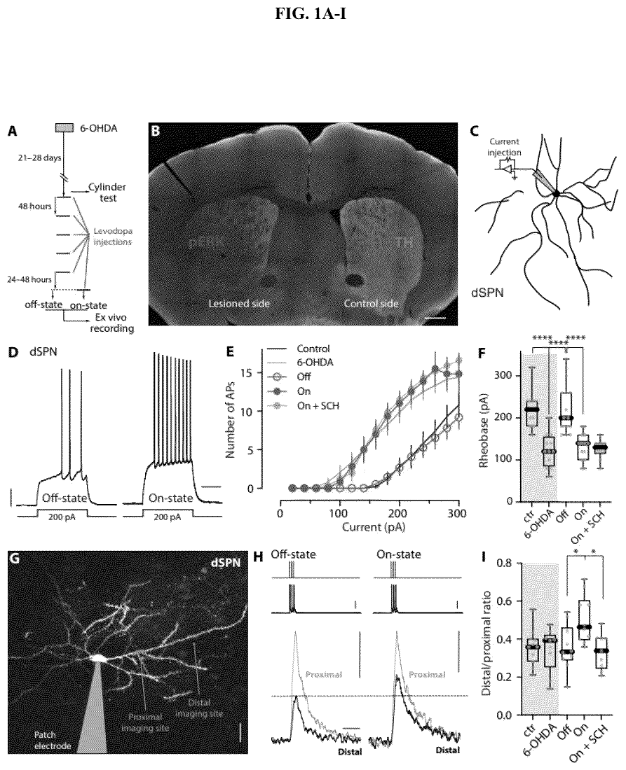

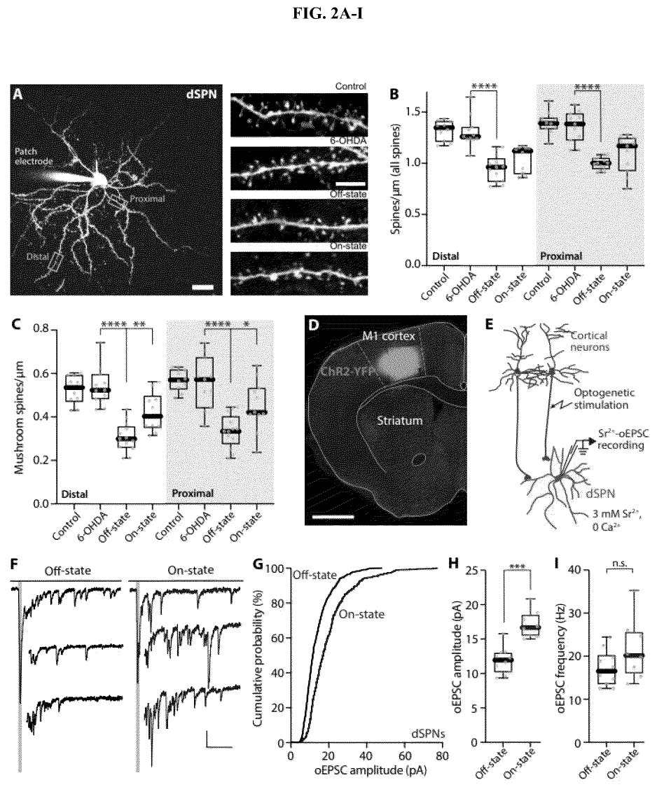

Intrinsic excitability and synaptic connectivity of dSPNs increased in the on-state of LID compared to off-state. Previous studies of how the induction of LID in rodents alters the intrinsic excitability and synaptic connectivity of SPNs have focused on the ‘off-state’ (usually 24-48 hours after the last administration of levodopa) (Fieblinger et al. 2014; Fieblinger et al. 2018; Nishijima et al. 2014; Picconi et al. 2003). The implicit assumption of these studies was that while SPN properties change over the days during the induction of LID, they do not change in the relatively short period after termination of levodopa treatment. To test this, the properties of dSPNs were compared in the off-state and on-state of LID. Briefly, Drd1-Tdtomato bacterial artificial chromosome (BAC) transgenic mice were rendered parkinsonian by unilateral, medial forebrain bundle (MFB) injection of 6-OHDA (FIG. 1A). Three to four weeks later, the extent of DA denervation was assessed using a drug-free, forelimb-use asymmetry test (also called cylinder test). Mice with a near-complete lesion were given dyskinesiogenic doses of levodopa every other day for at least five sessions (6 mg/kg for the first two sessions and 12 mg/kg for later sessions, supplemented with 12 mg/kg benserazide) (Cenci et al. 2018; Shen et al. 2016; Fieblinger et al. 2014). Mice were then sacrificed either 30 minutes after the last levodopa dose (on-state) or 24-48 hours after the last dose (off-state). In the on-state, phosphorylated ERK (PERK) in the DA-depleted striatum was elevated (FIG. 1B) (Santini et al. 2007; Fasano et al. 2010).

To assess somatic excitability, visually identified dSPNs in brain slices were subjected to whole-cell patch clamp recording (FIG. 1C). The response to current steps was monitored and the relationship between step amplitude and evoked spiking (F-I relationship) plotted (FIGS. 1, D and E). Similar to what has been described in previous studies, somatic excitability of dSPNs was significantly increased following 6-OHDA lesioning, and decreased by repetitive doses of levodopa when assessed in the off-state (both p<0.0001) (FIGS. 1, E and F). However, in brain slices taken shortly after levodopa treatment, dSPN somatic excitability was elevated. Specifically, in on-state dSPNs, the current needed to evoke a spike (rheobase) was significantly smaller than that in the off-state (p<0.0001) (FIG. 1F) and the number of spikes evoked by suprathreshold current steps was greater across a range of intensities (FIG. 1E). This shift in somatic excitability was consistent with there being an elevation in extracellular DA shortly after levodopa treatment, resulting in activation of DIRs on dSPNs. Furthermore, bath application of the DIR antagonist SCH 23390 (3 μM) did not reverse the leftward shift in the F-I relationship curve (FIG. 1E) or the reduction in rheobase (p=0.589) (FIG. 1F).

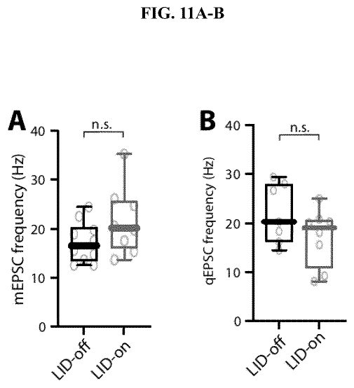

Most of the surface area of SPNs was composed of active dendrites, making them key sites of dopaminergic modulation. To assess dSPN dendritic excitability in on- and off-states, a combination of patch clamp electrophysiology and two-photon laser scanning microscopy (2PLSM) was used. Specifically, dSPNs in ex vivo brain slices from off- or on-state mice were patch clamped, filled with Ca²⁺-sensitive dye Fluo-4 and Ca²⁺-insensitive dye Alexa Fluor 568 to visualize dendrites, and injected with brief current steps (three 2 nA injections, 2 ms each, at 50 Hz); these somatically delivered steps evoked spikes that back-propagate into SPN dendrites (Day et al. 2008; Carrillo-Reid et al. 2019). To assess the spread of back-propagating action potentials (bAPs), 2PLSM was used to determine evoked changes in Fluo-4 fluorescence along dendrites produced by transient opening of voltage-dependent Ca²⁺ channels (Day et al. 2008; Carrillo-Reid et al. 2019). The magnitudes of the Ca²⁺ signals at proximal (~40 μm from soma) and distal (~90 μm from soma) dendritic locations served as a surrogate estimate of the extent of dendritic depolarization produced by the bAPs (FIG. 1G, H). To generate an estimate of bAP invasion that was independent of dye concentration, laser intensity and other experimental variables, the bAP-evoked Ca²⁺ signal in a distal dendritic segment was normalized by the bAP-evoked Ca²⁺ signal in the proximal region of the same dendrite. This index of dendritic excitability was significantly greater in on-state dSPNs than in off-state dSPNs (p=0.0355) (FIG. 11), as expected from an elevation in DIR signaling (Francardo & Cenci. 2014; Aubert et al. 2005). However, unlike somatic excitability, the elevation in on-state dSPN dendritic excitability was readily reversed by bath application of the DIR antagonist SCH 23390 (p=0.0295) (FIG. 1I).

In addition to modulating intrinsic excitability, dopaminergic signaling regulates the strength of glutamatergic synapses on dSPNs (Surmeier et al. 2011; Calabresi et al. 2007). Activation of DIRs promotes the induction of LTP at axospinous, corticostriatal synapses, which can manifest itself as a structural enlargement of spines (Shen et al. 2008; Yagishita et al. 2014). To determine LID-associated structural changes in dendritic spines, dSPNs in ex vivo brain slices were loaded with Alexa Fluor 568 through the patch pipette and then their dendritic morphology optically dissected using 2PLSM (FIG. 2A) (Fieblinger et al. 2014). Consistent with previous reports (Fieblinger et al. 2014; Fieblinger et al. 2018; Nishijima et al. 2014; Graves et al. 2019), dSPN spine density was not altered in either proximal or distal dendrites 3-4 weeks following unilateral 6-OHDA lesion (p=0.99 and 0.78 for distal and proximal dendrites, respectively) (FIG. 2, A-B). However, in dSPNs taken from off-state LID mice, spine density in both dendritic regions was significantly reduced (p<0.0001 in both proximal and distal dendrites) (FIG. 2, A-B). In dSPNs taken from on-state LID mice, spine density was not significantly different from that in the off-state (distal, p=0.1858; proximal, p=0.0597) (FIG. 2, A-B). However, in the on-state, enlarged, mushroom spines were more common in both distal and proximal dendrites (on-state vs. off-state: distal, p=0.0056; proximal, p=0.0066) (FIG. 2C).

To determine if these structural changes were accompanied by alterations in synaptic function, a combination of electrophysiological and optogenetic approaches was employed (Tanimura et al. 2019; Goda & Stevens. 1994; Perez-Rosello et al. 2019). To selectively activate corticostriatal synapses, an adeno-associated virus (AAV) carrying a channelrhodopsin 2 (ChR2) expression construct was injected into the motor cortex ipsilateral to the 6-OHDA lesion (FIG. 2D). In brain slices from off- and on-state Drd1-Tdtomato mice, dSPNs were patched with a Cs⁺-containing intracellular solution and voltage-clamped. To measure unitary synaptic strength, the extracellular Ca²⁺ was replaced with Sr2+ (3 mM), cortical fibers stimulated with blue LED pulses and asynchronous, excitatory postsynaptic currents (Sr²⁺-oEPSCs) recorded (FIG. 2, E-F). Consistent with the elevation in mushroom spine density, the distribution of Sr²⁺-oEPSC amplitudes was shifted toward larger values and the Sr²⁺-oEPSC amplitude was significantly larger in on-state dSPNs than in off-state dSPNs (p=0.0003) (FIG. 2, F-H). Although the frequency of on-state Sr²⁺-oEPSCs trended toward higher values (FIG. 21), as with spine density (FIG. 2, B-C), the differences between states were not statistically significant (p=0.127). Taken together, these observations suggest that on-state dopaminergic signaling in dyskinetic mice leads to an enhancement in intrinsic excitability and a potentiation of corticostriatal synaptic function in dSPNs, both of which are consistent with what is known about the consequences of DIR activation in dSPNs from healthy mice.

### Example 2

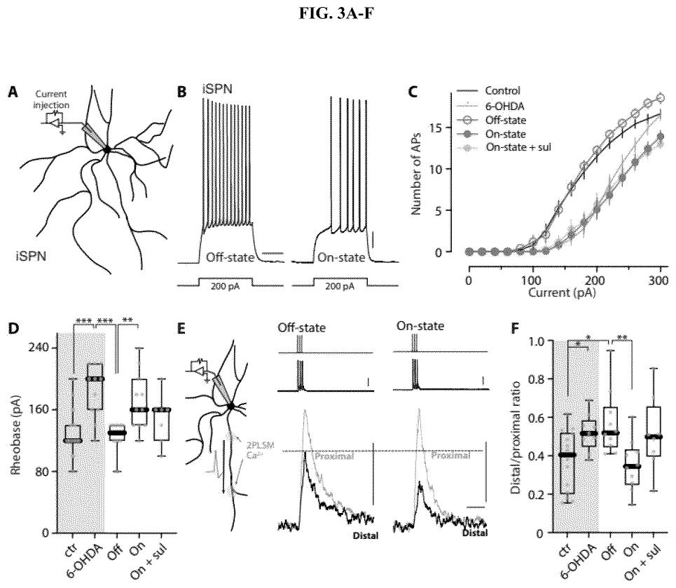

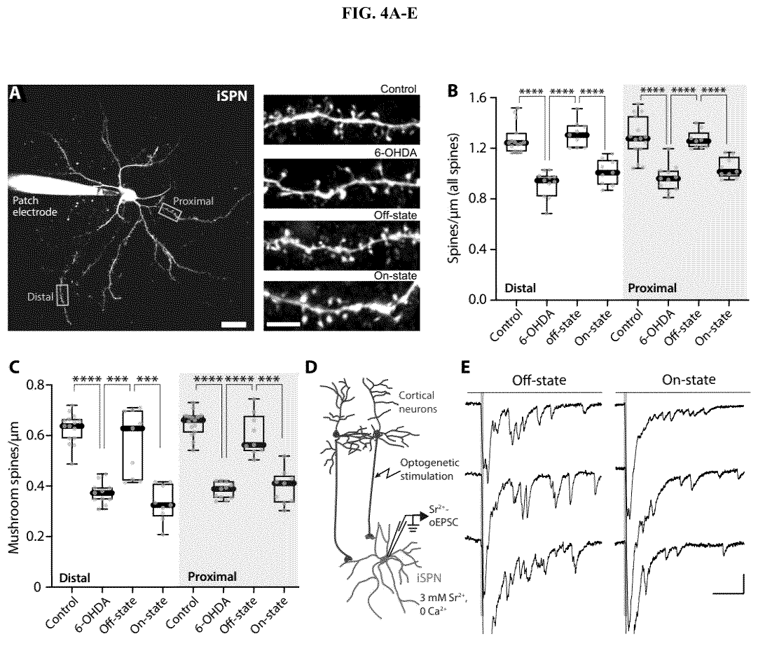

Intrinsic excitability and synaptic connectivity of iSPNs decreased in the on-state of LID. Although it is clear that dSPNs are critical to the emergence of LID, iSPNs also are pivotal to this condition. Clear changes in the properties of iSPNs following LID induction have been reported, but previous assessments have been limited to the off-state, leaving unexplored how they are modulated in the on-state. To fill this gap, iSPNs were studied using Drd2-enhanced green fluorescent protein (eGFP) mice following unilateral 6-OHDA lesioning and levodopa treatments. Somatic excitability was assessed using the same strategy as described above (FIG. 3, A-B). Somatic excitability of iSPNs was lowered by DA depletion and restored by repetitive doses of levodopa when assessed in the off-state (both p=0.002) (FIGS. 3, C and D). On-state iSPNs were found to be less excitable than those in the off-state (FIG. 3, B-D): rheobase was elevated compared to the off-state (p=0.0013) (FIG. 3D) and the F-I relationship curve shifted to the right (FIG. 3C). Acute antagonism of D2Rs with sulpiride (10 μM) in slices taken from on-state mice did not alter somatic excitability (rheobase, p=0.287), arguing that the hypoexcitability was not being maintained by DA (FIGS. 3, C and D).

To complement the assessment of somatic excitability, the dendritic excitability of iSPNs also was examined using the combination of patch clamp electrophysiology and 2PLSM, as described above. The index of dendritic excitability in iSPNs increased after DA depletion and stayed elevated after LID induction and termination of levodopa treatment (i.e., off-state) (control vs. 6-OHDA lesion, p=0.0327; control vs. off-state, p=0.0220). However, in the on-state, iSPN dendritic excitability fell (p=0.0029) (FIGS. 3, E and F). Acute blockade of dopaminergic signaling did not significantly alter dendritic excitability in on-state iSPNs (p=0.0545) (FIG. 3F), indicating some other factor was involved.

As with DIRs in dSPNs, D2R signaling in iSPNs modulates glutamatergic synaptic plasticity. Activation of D2Rs promotes the induction of LTD at axospinous, corticostriatal synapses, which in principle should lead to a reduction in spine size. In addition, homeostatic mechanisms have been posited to regulate iSPN spine density. As a first step toward determining whether there were alterations in spine density or size in the on-state, iSPNs in ex vivo brain slices from Drd2-eGFP mice were examined after 6-OHDA lesioning, LID induction and then termination of levodopa treatment (off-state). Spine density in both proximal and distal dendrites was significantly reduced 3-4 weeks after a 6-OHDA lesion (p<0.0001 in both proximal and distal dendritic locations) (FIGS. 4, A and B). After LID induction, off-state iSPN spine density returned to a range that was indistinguishable from that of iSPNs from mice without 6-OHDA lesions (FIGS. 4, A and B). In the on-state, the apparent spine density measured with 2PLSM fell significantly in both proximal and distal iSPN dendrites (on-state vs. off-state: p<0.0001 for both dendritic locations) (FIGS. 4, A and B), indicating that axospinous synapses were being added in the off-state and removed in the on-state. Similar changes were observed in the density of mushroom spines (on-state vs. off state: distal, p=0.0006; proximal, p=0.0002) (FIG. 4C).

If there were oscillations in the number of axospinous synapses between on- and off-states, then there should be a corresponding change in the frequency of asynchronous oEPSCs generated by activation of the corticostriatal pathway when extracellular Ca²⁺ was replaced with Sr2+. M1 cortical pyramidal neurons were induced to express ChR2 as described above and asynchronous, Sr²⁺-oEPSCs were recorded in iSPNs in ex vivo brain slices taken from mice in either the LID on- or off-states (FIGS. 4, D and E). The amplitude, but not the frequency, of Sr²⁺-oEPSCs was significantly smaller in on-state iSPNs than in off-state iSPNs (amplitude: p=0.0003; frequency: p=0.23) (FIG. 4, F-H). These observations are consistent with the inference that what is changing in iSPNs between on- and off-states is the strength, but not the number, of corticostriatal axospinous synapses.

Acaveat to the 2PLSM counts is that thin spines can be missed. Indeed, the density of SPN dendritic spines observed with high voltage electron microscopy (HVEM) is considerably greater than that estimated from three-dimensional 2PLSM reconstructions. Adora2-Cre mice were injected with a diluted AAV carrying a Cre-dependent eGFP expression construct (AAV9-pCAG-flex-EGFP-WPRE). This led to sparse labeling of iSPNs, allowing visualization of individual dendrites (FIGS. 4, I and J). Three-dimensional confocal reconstruction of iSPNs yielded spine density estimates in proximal dendrites that were significantly higher than those estimated from 2PLSM optical sectioning of live tissue (FIG. 4K) (confocal microscopy: median 2.084 vs. 2PLSM: median 1.243, spines per μm). After 6-OHDA lesioning, iSPN spine density estimated from confocal imaging decreased (control vs. 6-OHDA: p<0.0001), consistent with the 2PLSM estimates. Confocal spine density estimates did not return to control values in the off-state after LID induction, although they did increase modestly from the 6-OHDA lesioned state (p=0.0136) (FIGS. 4, J and K). As with 2PLSM, confocal estimates of iSPN spine density fell in the on-state (p=0.0001) (FIGS. 4, J and K), but only modestly.

These observations indicate that the apparent fluctuations in iSPN spine density following LID induction largely reflect alterations in the size- and detectability—but not the number of axospinous synapses. The distribution of spine diameters was hypothesized to be Gaussian (FIG. 4L). Given the limitations of light microscopy, there is a threshold diameter for spine detection; this threshold should be greater for 2PLSM which used a lens with relatively smaller numerical aperture (NA) than that used for the confocal analysis (0.9 NA 60× lens for 2PLSM, 1.49 NA 60× lens for confocal microscopy); hypothetical thresholds are drawn on the distributions to show how in principle they should change spine density estimates (FIG. 4L). After DA depletion, the distribution of spine diameters should change in two ways. First, the total number of synapses should drop by about 30% to reflect frank spine pruning. Second, the distribution of diameters should shift slightly toward larger values to account for the aberrant potentiation of axospinous synapses accompanying loss of iSPN D2R signaling (FIG. 4L). To illustrate the alterations between on- and off-states, the distribution of spine diameters was simply shifted to the right (larger) to reflect synaptic potentiation in the off-state and to the left (smaller) to reflect synaptic depression in the on-state (FIG. 4L). This simple model accounts for the apparent alterations in spine density estimated with 2PLSM, as well as the Sr²⁺-oEPSC data.

### Example 3

ACh release was elevated in PD and LID off-state and inhibited by D2R signaling. The data presented show that dyskinesiogenic fluctuations in dopaminergic signaling between the on- and off-states drive cell-specific changes in the intrinsic excitability and functional connectivity of iSPNs and dSPNs. While fluctuations in brain DA trigger these changes, it was unclear whether, at the level of individual SPNs, DA was acting alone. One key target of intrastriatal dopaminergic signaling was the ChI. Autonomously active ChIs are robustly inhibited by DA acting on D2Rs (Chantranupong et al. 2023; Chuhma et al. 2014; Straub et al. 2014; Wieland et al. 2014; Ding et al. 2010); moreover, ACh potently modulates the intrinsic excitability and synaptic plasticity of iSPNs and dSPNs through mechanisms that complement those of DA (Oldenburg et al. 2011; Zhai et al. 2023). However, the role of ChIs in the parkinsonian state and LID is controversial (Girasole et al. 2015; Shen et al. 2022; Bordia et al. 2016; Lim et al. 2014).

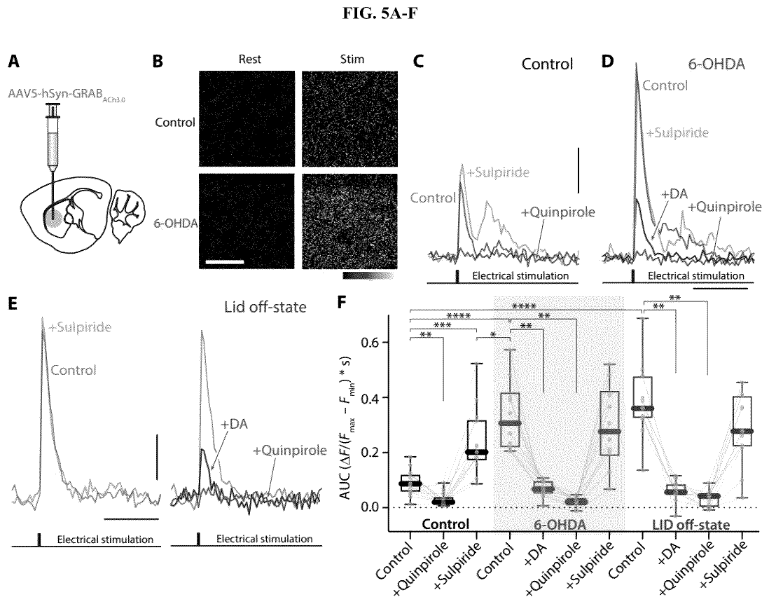

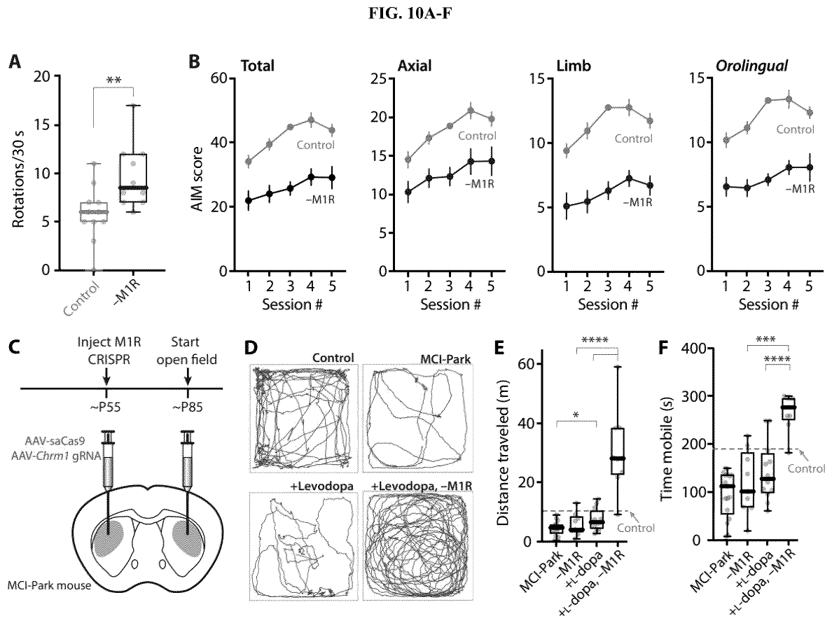

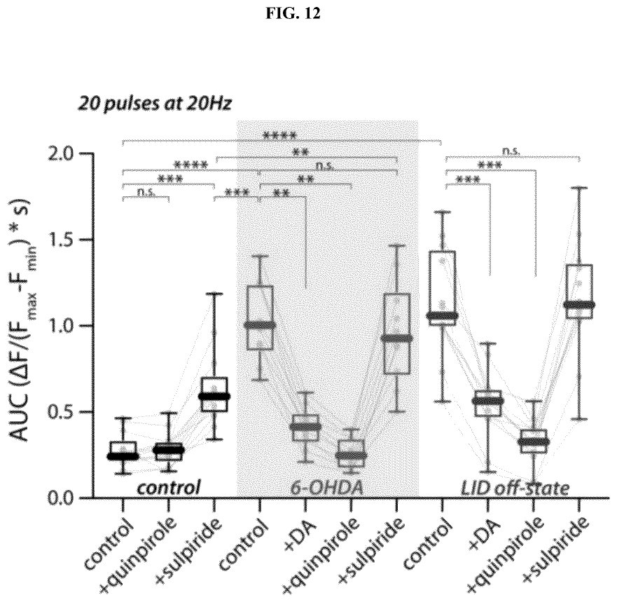

To directly assess the ACh release by ChIs in the parkinsonian and LID states, the genetically encoded ACh sensor GRABACh3.0 was used in conjunction with 2PLSM (Jing et al. 2020). About three weeks after stereotaxic injection into the dorsolateral striatum (DLS) of an AAV carrying a GRABACh3.0 expression construct, ex vivo brain slices were prepared and sensor fluorescence monitored in response to local electrical stimulation (FIG. 5, A-B). Robust ACh signals were evoked by either single pulses (FIG. 5) or short pulse bursts (FIG. 12). In the healthy DLS, the ACh signal rose rapidly and decayed back to baseline within 1-2 seconds (FIG. 5C). Bath application of the D2R antagonist sulpiride (10 μM) increased the evoked ACh signal (p=0.0005) (FIG. 5, C, F; FIG. 12), indicating that there was a basal dopaminergic tone. As expected, bath application of a D2R agonist quinpirole (10 μM) suppressed the ACh signal (p=0.0034) (FIG. 5, C, F; FIG. 12). In ex vivo slices from 6-OHDA lesioned mice, the GRABACh3.0 signal was dramatically elevated (unlesioned vs. 6-OHDA: p<0.0001) (FIG. 5, B,D,F; FIG. 10), suggesting that ACh release was disinhibited by DA depletion. Consistent with this scenario, bath application of sulpiride (10 μM) had no effect (p=0.5566), whereas D2R agonists (50 nM DA or 10 μM quinpirole) strongly inhibited ACh release in 6-OHDA mice (DA: p=0.002; quinpirole: p=0.002) (FIG. 5, D,F; FIG. 12). The peak GRABACh3.0 fluorescence in 6-OHDA-lesioned DLS was significantly greater than that in the control DLS in the presence of the D2R antagonist (p<0.0001), consistent with previous work suggesting that muscarinic autoreceptor function in ChIs was impaired following DA depletion (Ding et al. 2006). In slices from LID off-state mice, ACh release was significantly greater than that in the unlesioned striatum (p<0.0001), but similar to that following a 6-OHDA lesion (p=0.4679). Bath application of DA (50 nM), at a concentration comparable to that detected in the striatum in vivo during on-state (Lindgren et al. 2010; Miller & Abercrombie. 1999; Veronneau-Veilleux et al. 2021), strongly suppressed ACh release (p=0.001) (FIG. 5, E-F; FIG. 12). These data indicate that ACh release falls in the on-state as intrastriatal DA levels rise and then rebounds in the off-state when DA levels drop.

### Example 4

M1 muscarinic receptor signaling contributed to iSPN plasticity and LID induction. The intrinsic excitability and synaptic function of both iSPNs and dSPNs are potently modulated by muscarinic acetylcholine receptors (mAChRs). Previous work has implicated an imbalance in the activity of dSPN DIRs and M4Rs during the on-state in LID induction (Shen et al. 2016). How alterations in iSPN properties were influenced by disrupted cholinergic signaling was less clear-cut. In normal mice, activation of iSPN M1Rs enhances both somatic and dendritic excitability (Crittenden et al. 2021; Day et al. 2007; Shen et al. 2007). Genetic deletion of the gene coding for CDGI, which couples M1Rs to intracellular signaling pathways, also blunted the oxo-M-induced modulation of iSPNs dendritic integration and prevented induction of LTP at corticostriatal glutamatergic synapses (Crittenden et al. 2021).

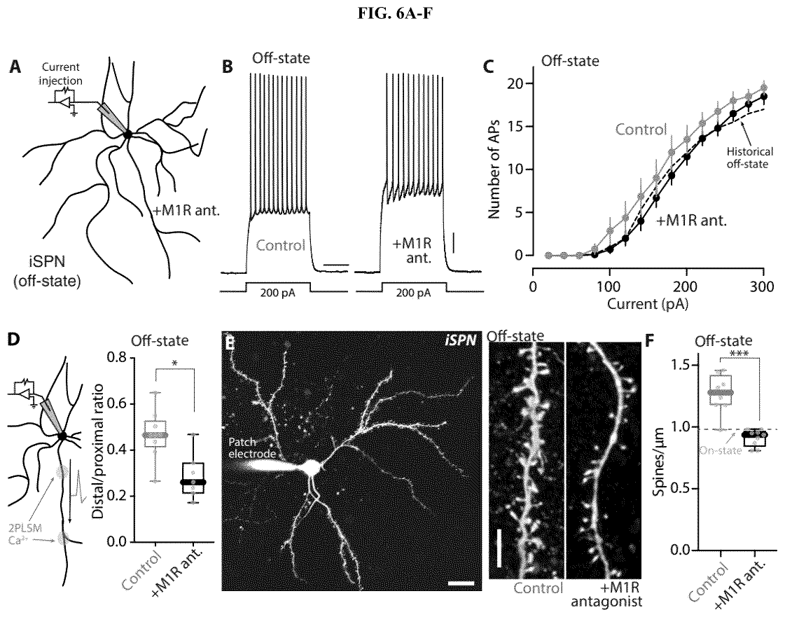

To assess the role of M1Rs in LID-induced adaptations in iSPNs, dyskinetic Drd2 BAC mice were given intraperitoneal injections of the M1R antagonist trihexyphenidyl hydrochloride (THP, 3 mg/kg) or saline at the beginning of the off-state (~2 hr after levodopa administration); THP is known to have good brain bioavailability and favorable pharmacokinetics after peripheral administration (Brocks et al. 1999). Mice were sacrificed 16 hours later, ex vivo brain slices prepared and iSPNs patch clamped (FIG. 6A). The slices from mice given THP were continuously incubated with the M1R selective antagonist VU 0255035 (5 μM) throughout the experiment. Somewhat unexpectedly, M1R antagonism did not have a significant impact on the elevation in iSPN somatic excitability in the off-state (FIG. 6, B-C). However, M1R antagonism did prevent the off-state elevation in dendritic excitability (p=0.035) (FIG. 6D) and the apparent elevation in spine density (p=0.0003) (FIG. 6, E-F).

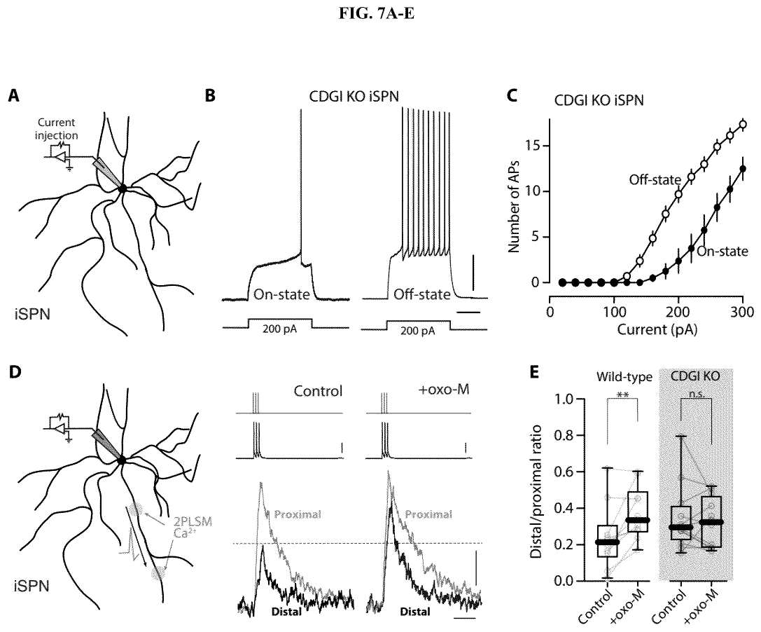

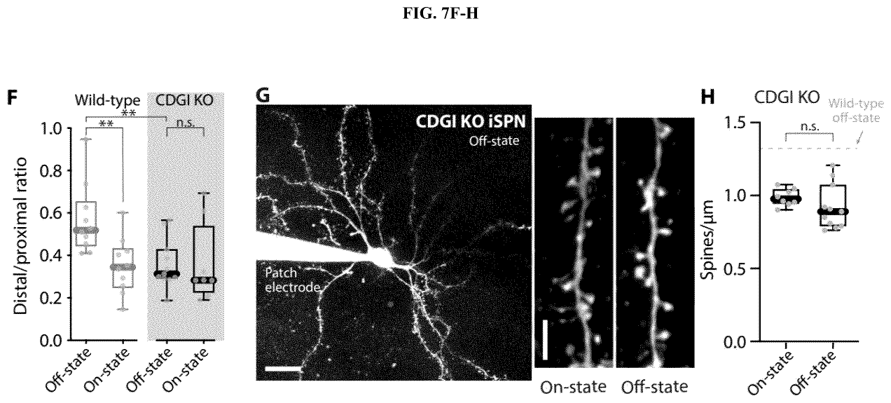

These observations indicate that M1R signaling contributes to the LID-induced adaptations in iSPNs, particularly those in the dendrites. As THP is not selective for M1Rs (Giachetti et al. 1986) and that M1Rs are broadly distributed in the brain, CDGI knockout (KO) mice were studied. CDGI is largely restricted to the striatum and is a key mediator of dendritic M1R signaling in iSPNs (Crittenden et al. 2021; Crittenden et al. 2009). In agreement with its dendritic role (Crittenden et al. 2021), deletion of CDGI did not blunt the oscillation in iSPN somatic excitability between LID on- and off-states (FIG. 7, A-C). To assess dendritic excitability, bAP propagation was examined. In agreement with previous work (Day et al. 2008), in ex vivo brain slices from naïve wildtype mice, bath application of the mAChR agonist oxo-M (10 μM) robustly increased the invasion of bAPs into the dendrites of iSPNs (p=0.0049) (FIG. 7, D-E). However, in ex vivo slices from naïve mice lacking CDGI, the M1R agonist oxo-M had no effect on dendritic invasion of bAPs (p=0.910) (FIG. 7, E). Consistent with this observation, the enhancement of bAP dendritic invasion seen in wildtype iSPNs in the LID off-state was not seen in CDGI KO iSPNs (off-state vs. on-state: wildtype, p=0.0205; CDGI KO, p=0.206) (FIG. 7F). Furthermore, the apparent increase in iSPN spine density observed in wildtype mice in the off-state (FIG. 4B) was not seen in iSPNs from CDGI KO mice (off-state vs. on-state: CDGI KO, p=0.613) (FIG. 7, G-H). Taken together, these observations suggest that dendritic M1R signaling in iSPNs plays a key role in the structural and functional adaptations accompanying the off-state in dyskinetic mice.

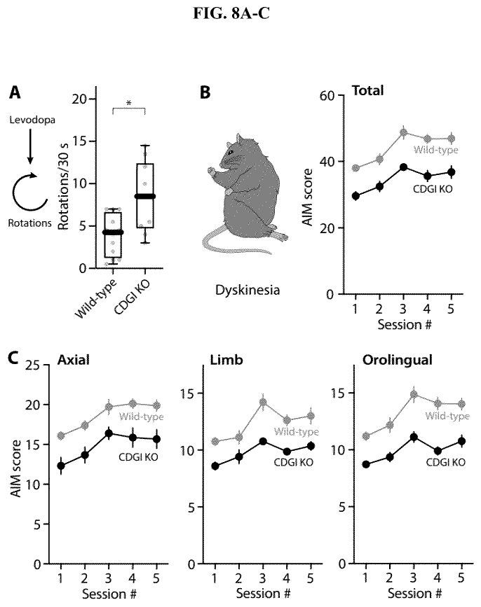

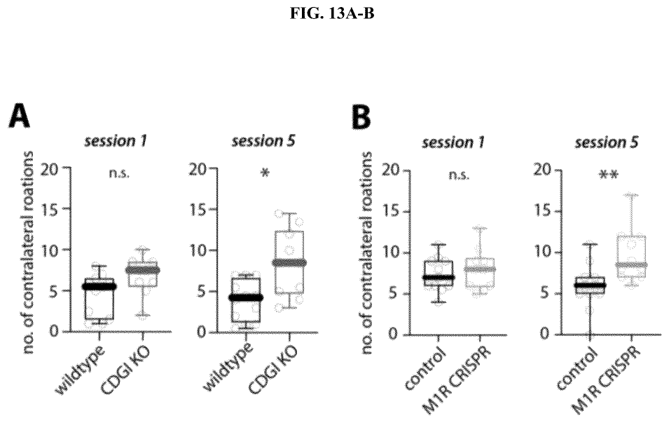

To determine if these CDGI-dependent adaptations were linked to behavior, the dyskinesia induced in wildtype and KO mice was compared using the protocol described above. CDGI KO and wildtype controls were unilaterally lesioned with 6-OHDA and about a month later evaluated for the extent of the lesion using the cylinder test. Mice with near-complete lesions were then injected with dyskinesiogenic doses of levodopa every other day and their abnormal involuntary movements (AIMs) were rated using an established rating scale (Cenci et al. 2007; Shen et al. 2016; Fieblinger et al. 2014). Routinely, the pro-kinetic effects of levodopa were assessed by monitoring the number of contralateral rotations in a 30 second period during peak-dose dyskinesia (Schwarting & Huston. 1996). The number of contralateral rotations induced by levodopa was significantly greater in CDGI KO mice than in wildtypes (p=0.0092 at the fifth dose) (FIG. 8A, FIG. 13A). Axial, limb and orolingual AIMs, as well as total AIMs, were less severe in CDGI KO mice than wildtype controls (FIGS. 8, B and C). These results indicate that CDGI-dependent M1R signaling in iSPN dendrites makes an important contribution to the network pathophysiology underlying LID.

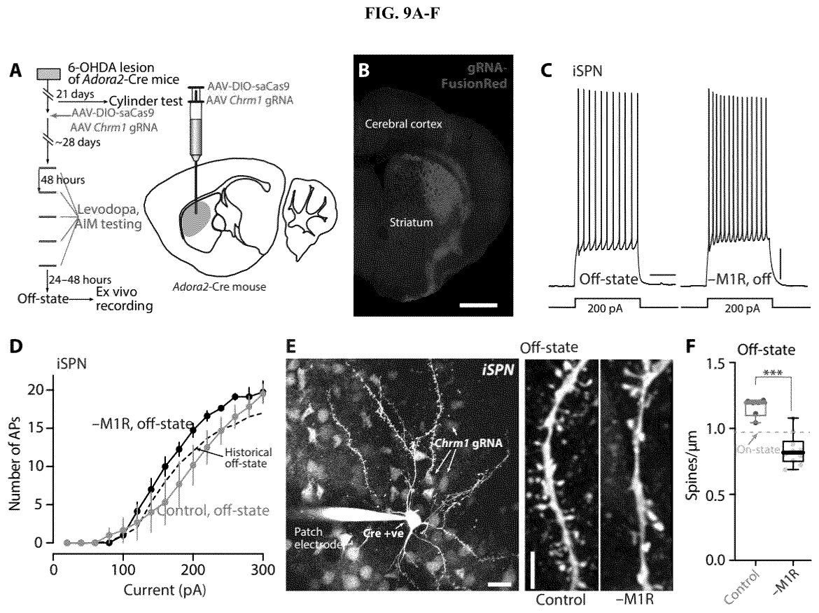

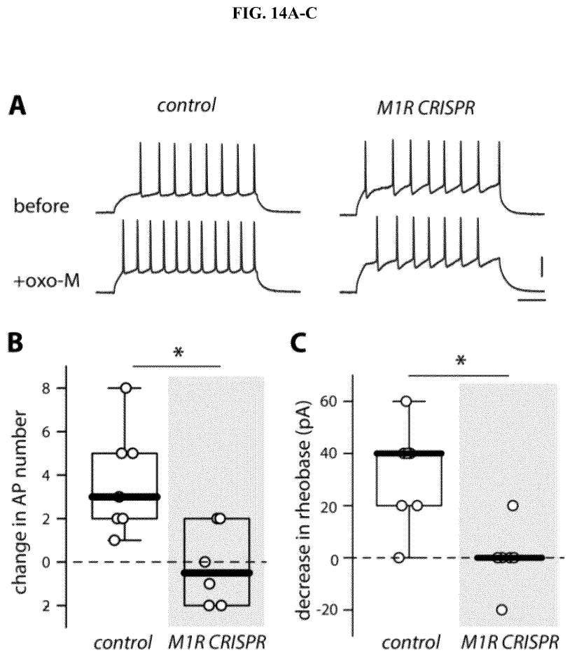

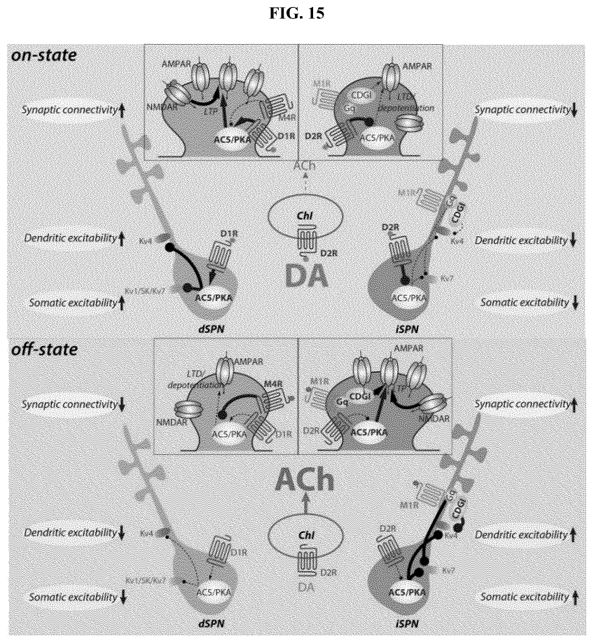

Although these results clearly implicate iSPNs in LID, it was possible that the loss of CDGI in other cell types (e.g., dSPNs) was a factor in the attenuation of LID. To address this caveat, a CRISPR-Cas9 approach was used to disrupt the expression of M1R specifically in iSPNs. In short, a mixture of two AAVs were injected into six sites across the ipsilateral DLS of unilaterally 6-OHDA lesioned Adora2-Cre mice that had passed the cylinder test (FIG. 9A); one of the AAVs carried a Cre-dependent Cas9 expression construct and the other carried Chrm1-targeting guide ribonucleic acid sequences (gRNAs; TGAGCTCTGTGTTGACCTTGA (SEQ ID NO: 1) and TCCCTGGCAAGTGGCATTCAT (SEQ ID NO: 2)) with a FusionRed (FR) reporter construct. Control mice were injected with just the gRNA-FR vector (FIG. 9A). In a subset of experiments where ex vivo analysis was to be performed, a third AAV carrying a Cre-dependent enhanced yellow fluorescent protein (eYFP) reporter was added. Roughly a month later, mice were subjected to the LID induction protocol (FIG. 9A). The AAVs expressed well (FIG. 9B) and the ability of the mAChR agonist oxo-M to enhance somatic excitability of iSPNs patch clamped in ex vivo brain slices was consistently lost (FIG. 14), demonstrating the efficacy of M1R deletion by the CRISPR-Cas9 approach.

To determine the cellular consequences of M1R deletion after LID induction, visually identified iSPNs in ex vivo brain slices were studied with patch clamp and 2PLSM approaches. As predicted from the systemic administration of the M1R antagonist (FIG. 6, B-C), genetic deletion of M1Rs from iSPNs did not alter the enhancement of somatic excitability observed in the off-state after LID induction (FIG. 9, C-D). Additionally, the dendritic architecture of iSPNs was examined by 2PLSM optical sectioning (FIG. 9, E-F). In agreement with the experiments employing systemic M1R antagonists or genetic deletion of CDGI, the apparent increase in iSPN spine density in the off-state following LID induction was blunted by genetic deletion of M1Rs (p=0.0005) (FIG. 9, E-F). Behavioral analysis of mice in which M1Rs had been deleted selectively from iSPNs yielded results that were similar to those obtained from CDGI KO mice. First, the therapeutic benefit of levodopa (as assessed by contralateral rotations) rose as dyskinesia developed in mice lacking M1Rs in iSPNs (FIG. 10A). Second, axial, limb and orolingual AIMs were dramatically attenuated by deletion of M1Rs in iSPNs (FIG. 10B). These results, show that dendritic M1R signaling in iSPNs contributes to structural and functional adaptations that cause the striatal pathophysiology underlying LID.

### Example 5

In both dSPNs and iSPNs, there was a profound oscillation in both intrinsic excitability and synaptic connectivity between on- and off-states following induction of LID in a mouse model of PD. These cellular and circuitry changes were complementary in nature, reinforcing the idea that an imbalance in the activity of dSPN and iSPN ensembles contributed to LID severity. Second, in parkinsonian and off-state dyskinetic mice there was a dramatic elevation in evoked ACh release, reflecting the loss of inhibitory D2R signaling. However, D2R signaling continued to robustly inhibit ACh release in tissue from dyskinetic mice, arguing that the oscillation in striatal DA concentrations associated with LID on- and off-states was mirrored by a counter-oscillation in ACh concentration. Third, the impact of cholinergic signaling on LID-associated adaptations in SPNs was particularly prominent in iSPNs. Indeed, blunting M1R signaling in iSPNs not only attenuated the oscillations in dendritic excitability and synaptic strength, but it also had clear behavioral effects-increasing the symptomatic benefit of levodopa and attenuating on-state dyskinesia.

### Example 6

LID was associated with cell- and state-specific adaptations, particularly in dendrites. Although previous studies have shown that LID induction was accompanied by physiological and anatomical changes in SPNs, most of the studies have focused on alterations that are evident in the off-state, long after the last levodopa dose (Fieblinger et al. 2014; Fieblinger et al. 2018; Nishijima et al. 2014; Picconi et al. 2003). An implicit assumption of many of these studies was that there are unlikely to be major changes in the relatively short period (hours) after levodopa treatment. This data shows that this assumption was incorrect.

In dSPNs recorded in ex vivo brain slices taken from mice shortly after their last dose of levodopa (on-state), somatic and dendritic excitability was significantly greater than in dSPNs recorded in slices taken from off-state mice. This observation was not surprising given the ability of DA acting at DIRs to increase the excitability of dSPNs at membrane potentials near spike threshold (Plotkin et al. 2011; Hernandez-Lopez et al. 1997; Surmeier et al. 1995; Wickens & Wilson. 1998). Interestingly, the augmentation in somatic excitability did not readily reverse with antagonism of DIRs, in keeping with the observation that in perforated patch recordings, this modulation was relatively persistent (Lahiri & Bevan. 2020). In contrast, the elevation in dendritic excitability was more time locked to DIR signaling, as it was acutely reversed by an antagonist. The difference in offset kinetics likely involves the regulation of protein phosphatases that reverse the covalent modifications to proteins achieved by DIR activation of protein kinase A (PKA) (Surmeier et al. 1995). Accompanying these changes in excitability were state-dependent alterations in synaptic function. As previously reported, the density of 2PLSM-visible spines on dSPN dendrites fell with LID induction and termination of levodopa (Fieblinger et al. 2014).

Additionally, right after levodopa treatment, the density of detectable spines, particularly those with a mushroom morphology, rose in dSPNs—as might be predicted by engagement of D1Rs and the induction of LTP at axospinous, glutamatergic synapses (Shen et al. 2008; Yagishita et al. 2014). Consistent with this inference, the amplitude of cortically evoked Sr²⁺-oEPSCs rose during the on-state. Both of these measures of synaptic strength reversed in the off-state, suggesting that the loss of dopaminergic signaling led to depotentiation or LTD (Shen et al. 2016; Picconi et al. 2003). Importantly, the frequency of Sr²⁺-oEPSCs did not change between states, suggesting that there was not a significant change in the number of synapses between on- and off-states. From a network perspective, it was important to note that this drug-induced oscillation in synaptic strength was not driven by the usual factors governing DA release and engagement of DIRs, like action initiation or reward. This suggests that there was a progressive randomization of corticostriatal synaptic strength with repeated levodopa treatment. If information that enables striatal dSPN ensembles to promote contextually appropriate action s stored in the strength of corticostriatal synapses, then the induction of LID should significantly degrade this information and the ability to move in a purposeful manner (Girasole et al. 2018; Ryan et al. 2024).

As with dSPNs, the properties of iSPNs in dyskinetic mice shifted between on- and off-states. In the on-state, somatic and dendritic excitability of iSPNs was lower than in the off-state. Unexpectedly, the suppression of iSPN excitability in the on-state did not readily reverse with D2R antagonism. One possibility mechanisms responsible for this persistence, was that on-state D2R signaling produces long-lasting alterations in ion channels governing excitability (i.e., intrinsic plasticity). In iSPNs, D2Rs could bring about this modulation through activation of phospholipase C and the protein phosphatase calcineurin, which regulates phosphorylation of a variety of ion channels, including Caᵥ1 Ca²⁺ channels (Hernandez-Lopez et aL. 2000). In contrast, the reasons for the elevation in off-state excitability of iSPNs are easier to surmise. Loss of ambient D2R signaling and disinhibition of ChIs undoubtedly contributed to this shift (Crittenden et al. 2021; Shen et al. 2007; Shen et al. 2005). Indeed, pharmacological antagonism of M1Rs or genetic deletion of CDGI, which mediates the dendritic signaling, blunted off-state dendritic excitability.

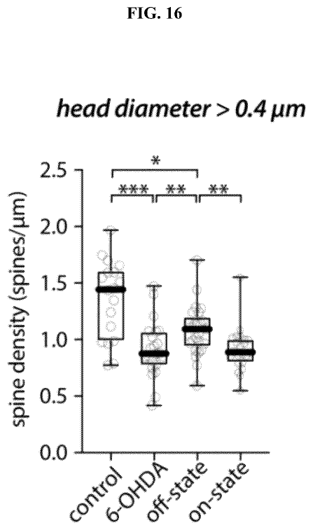

Another state-dependent change in iSPNs was in the strength of corticostriatal glutamatergic synapses. The amplitude of optogenetically evoked, asynchronous Sr²⁺-oEPSC in iSPNs fell during the on-state and rose during the off-state, consistent with the well described role of D2Rs in the modulation of long-term synaptic plasticity in these cells (Shen et al. 2008; Kreitzer et al. 2007; Kreitzer et al. 2005; Yin et al. 2006). Importantly, the frequency of asynchronous Sr²⁺-oEPSCs did not change between these states, arguing that the total number of synapses was unchanged. Although augmentation of synaptic strength in the off-state was consistent with previous studies (Fieblinger et al. 2018), the apparent stability in number of synapses was not. Previously, it was reported that iSPN spine density- and by inference synaptic density-increased in the off-state after the induction of dyskinesia (Fieblinger et al. 2014; Nishijima et al. 2014; Suarez et al. 2014). Although the estimates of spine density using 2PLSM rose in the off-state, reaching values close to those seen prior to 6-OHDA lesioning, higher-resolution confocal imaging of iSPN dendrites found only a modest change in spine density, well below values seen in controls. With the best optical approaches, the density of SPN spines in proximal dendrites was roughly 2.6 spines/μm, with many spines being less than a half micron in diameter (Parajuli et al. 2020; Wilson et al. 1983). Using confocal microscopy with a high numerical aperture (NA) lens (1.49), proximal spine density was estimated to be about 2 spines/μm, suggesting that about a quarter of the spines were being missed. Using 2PLSM with a lower NA lens (0.9), proximal spine density was estimated to be about 1.3 spines/μm, suggesting that roughly half of the spines were not being detected. The most parsimonious interpretation of the imaging and physiology was that what was changing between on- and off-states was not the number of spines, but rather their diameter and resolvability with optical methods. Indeed, when the confocal analysis was limited to large spines (>0.4 μm diameter) (FIG. 16), there was good alignment with the 2PLSM data. This was important because it suggests that iSPNs in an adult brain are not breaking and forming new synapses, but rather modulating their strength (which results in a size change). It was still the case, of course, that this modulation in synaptic strength was uncoupled from actions and their outcomes, unlike the situation in the normal striatum. Thus, this should result in a degradation in synaptic information and an impaired ability to properly coordinated movement.

### Example 7

Dysregulation of ChIs contributed to LID severity. In the healthy striatum, the interaction between DA and ACh release by ChIs plays a critical role in modulating the activity of SPNs and motor control (Chantranupong et al. 2023; Krok et al. 2023). In both dSPNs and iSPNs, this interaction is largely antagonistic, which creates a means of dynamically regulating basal ganglia output. In iSPNs, DA activation of D2Rs diminishes somatodendritic excitability and promotes LTD at glutamatergic synapses, whereas ACh activation of M1Rs does the opposite; in dSPNs, DIRs promote somatodendritic excitability and LTP at glutamatergic synapses whereas M4Rs do the opposite (Shen et al. 2016; Day et al. 2008; Shen et al. 2007; Hernandez-Lopez et al. 1997; Shen et al. 2008; Hernandez-Lopez et al. 2000; Shen et al. 2005). Complementing this interaction at the level of SPNs, ACh triggers DA release by activating nicotinic receptors on dopaminergic terminals (Cachope et al. 2012; Mohebi & Burke. 2023; Threlfell et al. 2012), whereas dopaminergic activation of D2Rs expressed by ChIs inhibits the autonomous spiking of ChIs and ACh release from axon terminals (Chantranupong et al. 2023; Chuhma et al. 2014; Straub et al. 2014; Wieland et al. 2014; Ding et al. 2006).

In PD, this dynamic interaction is disrupted. The loss of the striatal dopaminergic innervation in PD patients has long been thought to disinhibit ChIs, leading to a hyper-cholinergic state, a persistent imbalance in the excitability of iSPNs and dSPNs and ultimately bradykinesia (Albin et al. 1989; Laverne et al. 2022; Ding et al. 2009; Raz et al. 1996; Tanimura et al. 2019; Tubert et al. 2016; Kondabolu et al. 2016; Barrett et al. 2021). These data using the genetically encoded optical sensor of ACh (GRABACh3.0) were consistent with this premise, showing that in the 6-OHDA lesioned striatum, electrically evoked ACh release was significantly greater than in control striata. This upregulation was primarily attributable to the loss of presynaptic D2R signaling, as the D2R agonist quinpirole was able to almost eliminate evoked ACh release. That said, the fact that peak ACh release was significantly greater in the lesioned striatum than in the control striatum in the presence of the D2R antagonist sulpiride, also shows that mAChR autoreceptor function was impaired by DA depletion (Ding et al. 2006). Taken together, these data are consistent with the conclusion that ChIs play an important role in the pathophysiology underlying LID. Further evidence for this conclusion comes from the demonstration that disrupting M1R signaling in iSPNs, either by deleting CDGI globally or by CRISPR-Cas9-mediated deletion of Chrm1 specifically from them, blunted the induction of LID. Additionally, these results suggest that the engagement of ChIs and their remodeling of striatal circuitry was occurring primarily during the off-state.

### Example 7

Experiments are conducted to demonstrate that disrupting M1R expression in iSPNs by inhibitory nucleic acid (e.g., shRNA (CCTGGACTATGTGGCCAGCAA (SEQ ID NO: 3)) reduces and/or prevents LID without compromising the treatment with levodopa (e.g., enhancing levodopa treatment).

SEQEUNCES

SEQ ID NO: 1-M1 guide RNA-TGAGCTCTGTGTTGACCTTGA
SEQ ID NO: 2-M1 guide RNA-TCCCTGGCAAGTGGCATTCAT
SEQ ID NO: 3-M1 shRNA-CCTGGACTATGTGGCCAGCAA

### REFERENCES

The following references are herein incorporated by reference in their entireties.
Albin, R. L., Young, A. B. & Penney, J. B. The functional anatomy of basal ganglia disorders. Trends Neurosci 12, 366-375, doi:10.1016/0166-2236(89)90074-x (1989).
Cenci, M. A. Dopamine dysregulation of movement control in L-DOPA-induced dyskinesia. Trends Neurosci 30, 236-243, doi:10.1016/j.tins.2007.03.005 (2007).
Cenci, M. A. & Lindgren, H. S. Advances in understanding L-DOPA-induced dyskinesia. Curr Opin Neurobiol 17, 665-671, doi:10.1016/j.conb.2008.01.004 (2007).
Cenci, M. A. & Lundblad, M. Ratings of L-DOPA-induced dyskinesia in the unilateral 6-OHDA lesion model of Parkinson's disease in rats and mice. Curr Protoc Neurosci Chapter 9, Unit 9 25, doi:10.1002/0471142301.ns0925s41 (2007).
de la Fuente-Fernandez, R. et al. Levodopa-induced changes in synaptic dopamine levels increase with progression of Parkinson's disease: implications for dyskinesias. Brain 127, 2747-2754, doi:10.1093/brain/awh290 (2004).
Cenci, M. A., Jorntell, H. & Petersson, P. On the neuronal circuitry mediating L-DOPA-induced dyskinesia. J Neural Transm (Vienna) 125, 1157-1169, doi:10.1007/s00702-018-1886-0 (2018).
Zhai, S., Shen, W., Graves, S. M. & Surmeier, D. J. Dopaminergic modulation of striatal function and Parkinson's disease. J Neural Transm (Vienna) 126, 411-422, doi:10.1007/s00702-019-01997-y (2019).
Redgrave, P. et al. Goal-directed and habitual control in the basal ganglia: implications for Parkinson's disease. Nat Rev Neurosci 11, 760-772, doi:10.1038/nrn2915 (2010).
Gerfen, C. R. & Surmeier, D. J. Modulation of striatal projection systems by dopamine. Annu Rev Neurosci 34, 441-466, doi:10.1146/annurev-neuro-061010-113641 (2011).
Surmeier, D. J., Carrillo-Reid, L. & Bargas, J. Dopaminergic modulation of striatal neurons, circuits, and assemblies. Neuroscience 198, 3-18, doi:10.1016/j.neuroscience.2011.08.051 (2011).
Zhai, S., Tanimura, A., Graves, S. M., Shen, W. & Surmeier, D. J. Striatal synapses, circuits, and Parkinson's disease. Curr Opin Neurobiol 48, 9-16, doi:10.1016/j.conb.2017.08.004 (2018).
Tritsch, N. X. & Sabatini, B. L. Dopaminergic modulation of synaptic transmission in cortex and striatum. Neuron 76, 33-50, doi:10.1016/j.neuron.2012.09.023 (2012).
Surmeier, D. J., Ding, J., Day, M., Wang, Z. & Shen, W. D1 and D2 dopamine-receptor modulation of striatal glutamatergic signaling in striatal medium spiny neurons. Trends Neurosci 30, 228-235, doi:10.1016/j.tins.2007.03.008 (2007).
Berke, J. D. What does dopamine mean? Nat Neurosci 21, 787-793, doi:10.1038/s41593-018-0152-y (2018).
Chantranupong, L. et al. Dopamine and glutamate regulate striatal acetylcholine in decision-making. Nature 621, 577-585, doi:10.1038/s41586-023-06492-9 (2023).
Chuhma, N., Mingote, S., Moore, H. & Rayport, S. Dopamine neurons control striatal cholinergic neurons via regionally heterogeneous dopamine and glutamate signaling. Neuron 81, 901-912, doi:10.1016/j.neuron.2013.12.027 (2014).
Straub, C., Tritsch, N. X., Hagan, N. A., Gu, C. & Sabatini, B. L. Multiphasic modulation of cholinergic interneurons by nigrostriatal afferents. J Neurosci 34, 8557-8569, doi:10.1523/JNEUROSCI.0589-14.2014 (2014).
Wieland, S. et al. Phasic dopaminergic activity exerts fast control of cholinergic interneuron firing via sequential NMDA, D2, and D1 receptor activation. J Neurosci 34, 11549-11559, doi:10.1523/JNEUROSCI.1175-14.2014 (2014).19 Longo, F. et al. Cell-type-specific disruption of cortico-striatal circuitry drives repetitive patterns of behavior in fragile X syndrome model mice. Cell Rep 42, 112901, doi:10.1016/j.celrep.2023.112901 (2023).
Shen, W. X. et al. M4 Muscarinic Receptor Signaling Ameliorates Striatal Plasticity Deficits in Models of L-DOPA-Induced Dyskinesia (vol 88, pg 762, 2015). Neuron 90, 1139-1139, doi:10.1016/j.neuron.2016.05.017 (2016).
Laverne, G. et al. Cholinergic interneuron inhibition potentiates corticostriatal transmission in direct medium spiny neurons and rescues motor learning in parkinsonism. Cell Rep 40, 111034, doi:10.1016/j.celrep.2022.111034 (2022).
Crittenden, J. R. et al. CalDAG-GEFI mediates striatal cholinergic modulation of dendritic excitability, synaptic plasticity, and psychomotor behaviors. Neurobiol Dis 158, 105473, doi:10.1016/j.nbd.2021.105473 (2021).
Day, M., Wokosin, D., Plotkin, J. L., Tian, X. & Surmeier, D. J. Differential excitability and modulation of striatal medium spiny neuron dendrites. J Neurosci 28, 11603-11614, doi:10.1523/JNEUROSCI.1840-08.2008 (2008).
Shen, W. et al. Cholinergic modulation of Kir2 channels selectively elevates dendritic excitability in striatopallidal neurons. Nat Neurosci 10, 1458-1466, doi:10.1038/nn1972 (2007).
Crittenden, J. R. et al. Dysregulation of CalDAG-GEFI and CalDAG-GEFII predicts the severity of motor side-effects induced by anti-parkinsonian therapy. Proc Natl Acad Sci USA 106, 2892-2896, doi:10.1073/pnas.0812822106 (2009).
Girasole, A. E. & Nelson, A. B. Probing striatal microcircuitry to understand the functional role of cholinergic interneurons. Mov Disord 30, 1306-1318, doi:10.1002/mds.26340 (2015).
Ding, J. et al. RGS4-dependent attenuation of M4 auto receptor function in striatal cholinergic interneurons following dopamine depletion. Nat Neurosci 9, 832-842, doi:10.1038/nn1700 (2006).
Raz, A., Feingold, A., Zelanskaya, V., Vaadia, E. & Bergman, H. Neuronal synchronization of tonically active neurons in the striatum of normal and parkinsonian primates. J Neurophysiol 76, 2083-2088, doi:10.1152/jn.1996.76.3.2083 (1996).
Tanimura, A., Du, Y., Kondapalli, J., Wokosin, D. L. & Surmeier, D. J. Cholinergic Interneurons Amplify Thalamostriatal Excitation of Striatal Indirect Pathway Neurons in Parkinson's Disease Models. Neuron 101, 444-458 e446, doi 10.1016/j.neuron. 2018.12.004 (2019).
Tubert, C. et al. Decrease of a Current Mediated by Kv1.3 Channels Causes Striatal Cholinergic Interneuron Hyperexcitability in Experimental Parkinsonism. Cell Rep 16, 2749-2762, doi:10.1016/j.celrep.2016.08.016 (2016).
Maurice, N. et al. Striatal Cholinergic Interneurons Control Motor Behavior and Basal Ganglia Function in Experimental Parkinsonism. Cell Rep 13, 657-666, doi:10.1016/j.celrep.2015.09.034 (2015).
Kondabolu, K. et al. Striatal cholinergic interneurons generate beta and gamma oscillations in the corticostriatal circuit and produce motor deficits. Proc Natl Acad Sci USA 113, E3159-3168, doi:10.1073/pnas. 1605658113 (2016).
Barrett, M. J. et al. Antimuscarinic Anticholinergic Medications in Parkinson Disease: To Prescribe or Deprescribe? Mov Disord Clin Pract 8, 1181-1188, doi:10.1002/mdc3.13347 (2021).
Shen, W., Zhai, S. & Surmeier, D. J. Striatal synaptic adaptations in Parkinson's disease. Neurobiol Dis 167, 105686, doi:10.1016/j.nbd.2022.105686 (2022).35 Bordia, T., Perez, X. A., Heiss, J., Zhang, D. & Quik, M. Optogenetic activation of striatal cholinergic interneurons regulates L-dopa-induced dyskinesias. Neurobiol Dis 91, 47-58, doi:10.1016/j.nbd.2016.02.019 (2016).
Lim, S. A., Kang, U. J. & McGehee, D. S. Striatal cholinergic interneuron regulation and circuit effects. Front Synaptic Neurosci 6, 22, doi:10.3389/fnsyn.2014.00022 (2014).
Ding, Y. et al. Enhanced striatal cholinergic neuronal activity mediates L-DOPA-induced dyskinesia in parkinsonian mice. Proc Natl Acad Sci USA 108, 840-845, doi:10.1073/pnas. 1006511108 (2011).
Won, L., Ding, Y., Singh, P. & Kang, U. J. Striatal cholinergic cell ablation attenuates L-DOPA induced dyskinesia in Parkinsonian mice. J Neurosci 34, 3090-3094, doi:10.1523/JNEUROSCI.2888-13.2014 (2014).
Fieblinger, T. et al. Cell type-specific plasticity of striatal projection neurons in parkinsonism and L-DOPA-induced dyskinesia. Nat Commun 5, 5316, doi:10.1038/ncomms6316 (2014).
Fieblinger, T. et al. Striatonigral neurons divide into two distinct morphological-physiological phenotypes after chronic L-DOPA treatment in parkinsonian rats. Sci Rep 8, 10068, doi:10.1038/s41598-018-28273-5 (2018).
Nishijima, H. et al. Morphologic changes of dendritic spines of striatal neurons in the levodopa-induced dyskinesia model. Mov Disord 29, 336-343, doi:10.1002/mds.25826 (2014).
Picconi, B. et al. Loss of bidirectional striatal synaptic plasticity in L-DOPA-induced dyskinesia. Nat Neurosci 6, 501-506, doi:10.1038/nn1040 (2003).
Santini, E. et al. Critical involvement of cAMP/DARPP-32 and extracellular signal-regulated protein kinase signaling in L-DOPA-induced dyskinesia. J Neurosci 27, 6995-7005, doi:10.1523/JNEUROSCI.0852-07.2007 (2007).
Fasano, S. et al. Inhibition of Ras-guanine nucleotide-releasing factor 1 (Ras-GRF1) signaling in the striatum reverts motor symptoms associated with L-dopa-induced dyskinesia. Proc Natl Acad Sci USA 107, 21824-21829, doi:10.1073/pnas. 1012071107 (2010).
Zhai, S. et al. Ca (2+)-dependent phosphodiesterase 1 regulates the plasticity of striatal spiny projection neuron glutamatergic synapses. Cell Rep 43, 114540, doi:10.1016/j.celrep.2024.114540 (2024).
Plotkin, J. L., Day, M. & Surmeier, D. J. Synaptically driven state transitions in distal dendrites of striatal spiny neurons. Nat Neurosci 14, 881-888, doi:10.1038/nn.2848 (2011).
Hernandez-Lopez, S., Bargas, J., Surmeier, D. J., Reyes, A. & Galarraga, E. D1 receptor activation enhances evoked discharge in neostriatal medium spiny neurons by modulating an L-type Ca²⁺ conductance. J Neurosci 17, 3334-3342, doi:10.1523/JNEUROSCI.17-09-03334.1997 (1997).
Surmeier, D. J., Bargas, J., Hemmings, H. C., Jr., Nairn, A. C. & Greengard, P. Modulation of calcium currents by a D1 dopaminergic protein kinase/phosphatase cascade in rat neostriatal neurons. Neuron 14, 385-397, doi:10.1016/0896-6273(95)90294-5 (1995).
Wickens, J. R. & Wilson, C. J. Regulation of action-potential firing in spiny neurons of the rat neostriatum in vivo. J Neurophysiol 79, 2358-2364, doi:10.1152/jn. 1998.79.5.2358 (1998).
Francardo, V. & Cenci, M. A. Investigating the molecular mechanisms of L-DOPA-induced dyskinesia in the mouse. Parkinsonism Relat Disord 20 Suppl 1, S20-22, doi:10.1016/S1353-8020(13)70008-7 (2014).
Aubert, I. et al. Increased D1 dopamine receptor signaling in levodopa-induced dyskinesia. Ann Neurol 57, 17-26, doi:10.1002/ana.20296 (2005).52 Lahiri, A. K. & Bevan, M. D. Dopaminergic Transmission Rapidly and Persistently Enhances Excitability of D1 Receptor-Expressing Striatal Projection Neurons. Neuron 106, 277-290 e276, doi:10.1016/j.neuron.2020.01.028 (2020).
Carrillo-Reid, L. et al. Mutant huntingtin enhances activation of dendritic Kv4 K (+) channels in striatal spiny projection neurons. Elife 8, doi:10.7554/eLife.40818 (2019).
Calabresi, P., Picconi, B., Tozzi, A. & Di Filippo, M. Dopamine-mediated regulation of corticostriatal synaptic plasticity. Trends Neurosci 30, 211-219, doi:10.1016/j.tins.2007.03.001 (2007).
Shen, W., Flajolet, M., Greengard, P. & Surmeier, D. J. Dichotomous dopaminergic control of striatal synaptic plasticity. Science 321, 848-851, doi:10.1126/science.1160575 (2008).
Yagishita, S. et al. A critical time window for dopamine actions on the structural plasticity of dendritic spines. Science 345, 1616-1620, doi:10.1126/science. 1255514 (2014).
Graves, S. M. & Surmeier, D. J. Delayed Spine Pruning of Direct Pathway Spiny Projection Neurons in a Mouse Model of Parkinson's Disease. Front Cell Neurosci 13, 32, doi:10.3389/fncel.2019.00032 (2019).
Goda, Y. & Stevens, C. F. Two components of transmitter release at a central synapse. Proc Natl Acad Sci USA 91, 12942-12946, doi:10.1073/pnas.91.26.12942 (1994).
Perez-Rosello, T. et al. Enhanced striatopallidal gamma-aminobutyric acid (GABA) (A) receptor transmission in mouse models of huntington's disease. Mov Disord 34, 684-696, doi:10.1002/mds.27622 (2019).
Alcacer, C. et al. Chemogenetic stimulation of striatal projection neurons modulates responses to Parkinson's disease therapy. J Clin Invest 127, 720-734, doi:10.1172/JCI90132 (2017).
Shen, W. et al. Cell- and state-specific plasticity of striatal glutamatergic synapses is critical to the expression of levodopa-induced dyskinesia. bioRxiv, 2024.2006.2014.599055, doi:10.1101/2024.06.14.599055 (2024).
Kreitzer, A. C. & Malenka, R. C. Endocannabinoid-mediated rescue of striatal LTD and motor deficits in Parkinson's disease models. Nature 445, 643-647, doi:10.1038/nature05506 (2007).
Matsuzaki, M., Honkura, N., Ellis-Davies, G. C. & Kasai, H. Structural basis of long-term potentiation in single dendritic spines. Nature 429, 761-766, doi:10.1038/nature02617 (2004).
Day, M. et al. Selective elimination of glutamatergic synapses on striatopallidal neurons in Parkinson disease models. Nat Neurosci 9, 251-259, doi:10.1038/nn1632 (2006).
Suarez, L. M. et al. L-DOPA treatment selectively restores spine density in dopamine receptor D2-expressing projection neurons in dyskinetic mice. Biol Psychiatry 75, 711-722, doi:10.1016/j.biopsych.2013.05.006 (2014).
Parajuli, L. K. et al. Developmental Changes in Dendritic Spine Morphology in the Striatum and Their Alteration in an A53T alpha-Synuclein Transgenic Mouse Model of Parkinson's Disease. eNeuro 7, doi:10.1523/ENEURO.0072-20.2020 (2020).
Wilson, C. J., Groves, P. M., Kitai, S. T. & Linder, J. C. Three-dimensional structure of dendritic spines in the rat neostriatum. J Neurosci 3, 383-388, doi:10.1523/JNEUROSCI.03-02-00383.1983 (1983).
Ding, J. B., Guzman, J. N., Peterson, J. D., Goldberg, J. A. & Surmeier, D. J. Thalamic gating of corticostriatal signaling by cholinergic interneurons. Neuron 67, 294-307, doi:10.1016/j.neuron.2010.06.017 (2010).69 Oldenburg, I. A. & Ding, J. B. Cholinergic modulation of synaptic integration and dendritic excitability in the striatum. Curr Opin Neurobiol 21, 425-432, doi:10.1016/j.conb.2011.04.004 (2011).
Zhai, S., Cui, Q., Simmons, D. V. & Surmeier, D. J. Distributed dopaminergic signaling in the basal ganglia and its relationship to motor disability in Parkinson's disease. Curr Opin Neurobiol 83, 102798, doi:10.1016/j.conb.2023.102798 (2023).
Jing, M. et al. An optimized acetylcholine sensor for monitoring in vivo cholinergic activity. Nat Methods 17, 1139-1146, doi:10.1038/s41592-020-0953-2 (2020).
Lindgren, H. S., Andersson, D. R., Lagerkvist, S., Nissbrandt, H. & Cenci, M. A. L-DOPA-induced dopamine efflux in the striatum and the substantia nigra in a rat model of Parkinson's disease: temporal and quantitative relationship to the expression of dyskinesia. J Neurochem 112, 1465-1476, doi:10.1111/j.1471-4159.2009.06556.x (2010).
Miller, D. W. & Abercrombie, E. D. Role of high-affinity dopamine uptake and impulse activity in the appearance of extracellular dopamine in striatum after administration of exogenous L-DOPA: studies in intact and 6-hydroxydopamine-treated rats. J Neurochem 72, 1516-1522, doi:10.1046/j.1471-4159.1999.721516.x (1999).
Veronneau-Veilleux, F., Robaey, P., Ursino, M. & Nekka, F. An integrative model of Parkinson's disease treatment including levodopa pharmacokinetics, dopamine kinetics, basal ganglia neurotransmission and motor action throughout disease progression. J Pharmacokinet Pharmacodyn 48, 133-148, doi:10.1007/s10928-020-09723-y (2021).
Brocks, D. R. Anticholinergic drugs used in Parkinson's disease: An overlooked class of drugs from a pharmacokinetic perspective. J Pharm Sci 2, 39-46 (1999).
Giachetti, A., Giraldo, E., Ladinsky, H. & Montagna, E. Binding and functional profiles of the selective M1 muscarinic receptor antagonists trihexyphenidyl and dicyclomine. Br J Pharmacol 89, 83-90, doi:10.1111/j.1476-5381.1986.tb11123.x (1986).
Schwarting, R. K. & Huston, J. P. The unilateral 6-hydroxydopamine lesion model in behavioral brain research. Analysis of functional deficits, recovery and treatments. Prog Neurobiol 50, 275-331, doi:10.1016/s0301-0082(96) 00040-8 (1996).
Choi, S. J. et al. Alterations in the intrinsic properties of striatal cholinergic interneurons after dopamine lesion and chronic L-DOPA. Elife 9, doi:10.7554/eLife.56920 (2020).
Girasole, A. E. et al. A Subpopulation of Striatal Neurons Mediates Levodopa-Induced Dyskinesia. Neuron 97, 787-795 e786, doi:10.1016/j.neuron.2018.01.017 (2018).
Ryan, M. B. et al. Excessive firing of dyskinesia-associated striatal direct pathway neurons is gated by dopamine and excitatory synaptic input. Cell Rep 43, 114483, doi:10.1016/j.celrep.2024.114483 (2024).
Debanne, D., Inglebert, Y. & Russier, M. Plasticity of intrinsic neuronal excitability. Curr Opin Neurobiol 54, 73-82, doi:10.1016/j.conb.2018.09.001 (2019).
Hansel, C. & Yuste, R. Neural ensembles: role of intrinsic excitability and its plasticity. Front Cell Neurosci 18, 1440588, doi:10.3389/fncel.2024.1440588 (2024).
Hernandez-Lopez, S. et al. D2 dopamine receptors in striatal medium spiny neurons reduce L-type Ca²⁺ currents and excitability via a novel PLC [beta]1-IP3-calcineurin-signaling cascade. J Neurosci 20, 8987-8995, doi:10.1523/JNEUROSCI.20-24-08987.2000 (2000).
Shen, W., Hamilton, S. E., Nathanson, N. M. & Surmeier, D. J. Cholinergic suppression of KCNQ channel currents enhances excitability of striatal medium spiny neurons. J Neurosci 25, 7449-7458, doi:10.1523/JNEUROSCI. 1381-05.2005 (2005).
Kreitzer, A. C. & Malenka, R. C. Dopamine modulation of state-dependent endocannabinoid release and long-term depression in the striatum. J Neurosci 25, 10537-10545, doi:10.1523/JNEUROSCI.2959-05.2005 (2005).86 Yin, H. H. & Lovinger, D. M. Frequency-specific and D2 receptor-mediated inhibition of glutamate release by retrograde endocannabinoid signaling. Proc Natl Acad Sci USA 103, 8251-8256, doi:10.1073/pnas.0510797103 (2006).
Krok, A. C. et al. Intrinsic dopamine and acetylcholine dynamics in the striatum of mice. Nature 621, 543-549, doi:10.1038/s41586-023-05995-9 (2023).
Cachope, R. et al. Selective activation of cholinergic interneurons enhances accumbal phasic dopamine release: setting the tone for reward processing. Cell Rep 2, 33-41, doi:10.1016/j.celrep.2012.05.011 (2012).
Mohebi, A., Collins, V. L. & Berke, J. D. Accumbens cholinergic interneurons dynamically promote dopamine release and enable motivation. Elife 12, doi:10.7554/eLife.85011 (2023).
Threlfell, S. et al. Striatal dopamine release is triggered by synchronized activity in cholinergic interneurons. Neuron 75, 58-64, doi:10.1016/j.neuron.2012.04.038 (2012).
Gangarossa, G. et al. Role of the atypical vesicular glutamate transporter VGLUT3 in 1-DOPA-induced dyskinesia. Neurobiol Dis 87, 69-79, doi:10.1016/j.nbd.2015.12.010 (2016).
Castello, J. et al. The Dopamine D5 receptor contributes to activation of cholinergic interneurons during L-DOPA induced dyskinesia. Sci Rep 10, 2542, doi:10.1038/s41598-020-59011-5 (2020).
Paz, R. M. et al. Levodopa Causes Striatal Cholinergic Interneuron Burst-Pause Activity in Parkinsonian Mice. Mov Disord 36, 1578-1591, doi:10.1002/mds.28516 (2021).
Citri, A. & Malenka, R. C. Synaptic plasticity: multiple forms, functions, and mechanisms. Neuropsychopharmacology 33, 18-41, doi:10.1038/sj.npp 1301559 (2008).
Holtmaat, A. & Svoboda, K. Experience-dependent structural synaptic plasticity in the mammalian brain. Nat Rev Neurosci 10, 647-658, doi:10.1038/nrn2699 (2009).
Surmeier, D. J., Plotkin, J. & Shen, W. Dopamine and synaptic plasticity in dorsal striatal circuits controlling action selection. Curr Opin Neurobiol 19, 621-628, doi:10.1016/j.conb.2009.10.003 (2009).
Cheung, T. H. C., Ding, Y., Zhuang, X. & Kang, U. J. Learning critically drives parkinsonian motor deficits through imbalanced striatal pathway recruitment. Proc Natl Acad Sci USA 120, e2213093120, doi:10.1073/pnas.2213093120 (2023).
Alagarsamy, S. et al. Activation of NMDA receptors reverses desensitization of mGluR5 in native and recombinant systems. Nat Neurosci 2, 234-240, doi:10.1038/6338 (1999).
Jin, D. Z., Guo, M. L., Xue, B., Mao, L. M. & Wang, J. Q. Differential regulation of CaMKIIalpha interactions with mGluR5 and NMDA receptors by Ca (2+) in neurons. J Neurochem 127, 620-631, doi:10.1111/jnc. 12434 (2013).
Abraham, W. C. & Bear, M. F. Metaplasticity: the plasticity of synaptic plasticity. Trends Neurosci 19, 126-130, doi:10.1016/s0166-2236 (96) 80018-x (1996).
Bear, M. F. Mechanism for a sliding synaptic modification threshold. Neuron 15, 1-4, doi:10.1016/0896-6273 (95) 90056-x (1995).
Lee, H. K. & Kirkwood, A. Mechanisms of Homeostatic Synaptic Plasticity in vivo. Front Cell Neurosci 13, 520, doi:10.3389/fncel.2019.00520 (2019).
Hunker, A. C. et al. Enhancer AAV toolbox for accessing and perturbing striatal cell types and circuits. bioRxiv, doi:10.1101/2024.09.27.615553 (2024).

1. A method of treating Levodopa-induced dyskinesia (LID), in a subject in need thereof, the method comprising: administering a M1 muscarinic acetylcholine receptor (M1R) antagonist to the subject.

2. The method of claim 1, wherein the M1R antagonist is administered into an indirect pathway spiny projection neuron (iSPN).

3. The method of claim 1, wherein the M1R antagonist selectively disrupts M1 muscarinic acetylcholine receptor signaling.

4. The method of claim 3, wherein the M1R antagonist is a nucleic acid inhibitor of M1 muscarinic acetylcholine expression.

5. The method of claim 4, wherein the nucleic acid inhibitor of M1 muscarinic acetylcholine expression knocks down the expression of iSPN M1Rs to suppress cholinergic signaling to attenuate oscillations in dendritic excitability and synaptic strength.

6. The method of claim 1, wherein the nucleic acid inhibitor of M1 muscarinic acetylcholine expression is a microRNA (miRNA), a small interfering (siRNA), a short hairpin RNA (shRNA), an anti-sense RNA (asRNA), a competing endogenous RNA (ceRNA), a long non-coding RNA (lncRNA) and a ribozyme.

7. The method of claim 4, wherein the nucleic acid inhibitor of M1 muscarinic acetylcholine expression is delivered with a vector.

8. The method of claim 7, wherein the vector is a viral vector.

9. The method of claim 8, wherein the viral vector is an adeno-associated viral vector (AAV), a adenoviral vector, a lentiviral vector, a non-viral vector, a human-compatible vector, a herpes simplex virus vector and a retroviral vector.

10. The method of claim 1, wherein the subject is afflicted with the neurodegenerative disease associated with intrinsic and synaptic changes in striatal spiny projection neurons (SPNs) triggered by the sustained depletion of dopamine (DA).

11. The method of claim 10, wherein the neurodegenerative disease is Parkinson's disease.

12. The method of claim 1, wherein the subject is a human.

13. The method of claim 1, wherein the subject was administered or is currently being administered by a therapeutic agent.

14. The method of claim 13, wherein the therapeutic agent administered is levodopa (L-3,4-dihydroxyphenylalanine).

15. The method of claim 14, wherein administering levodopa to the subject induces fluctuations of DA levels in a subject; wherein large and uncontrolled fluctuations in DA levels results in LID.

16. The method of claim 15, wherein administering levodopa to the subject increases acetylcholine release in the subject's off-state.

17. The method of claim 16, wherein the increased acetylcholine release in the off-state results in the upregulation of oscillations in dendritic excitability and synaptic strength.

18. The method of claim 1, wherein the M1R antagonist reduces the function of iSPN M1Rs to diminish the induction and expression of LID.

19. The method of claim 1, wherein the M1R antagonist is administered by an Intraperitoneal (IP) injection.

20-22. (canceled)

23. The method of claim 10, wherein said M1R antagonist is administered during the low levels of DA to the subject after levodopa is administered.

24-27. (canceled)
